(19) United States
(12) Patent Application Publication
(10) Pub. No.: US 2026/0279571 A1
(43) Pub. Date: Sep. 17, 2026

(54) IMPEDANCE-BASED CLASSIFICATION OF BIOLOGICAL CELLS OR OTHER PARTICLES VIA MACHINE LEARNING METHODS

(71) Applicant: University of Puerto Rico, San Juan, PR (US)

(72) Inventors: Rubén E. Diaz-Rivera, San Juan, PR (US); Marco A. Becerra Arias, Utuado, PR (US); Malcom Díaz Garcia, San Juan, PR (US); Euginio Caraballo-Justiniano, San Juan, PR (US)

(73) Assignee: University of Puerto Rico, San Juan, PR (US)

(21) Appl. No.: 19/491,117

(22) PCT Filed: Apr. 9, 2024
(86) PCT No.: PCT/US2024/023701

§ 371 Date: Dec. 8, 2025

Related U.S. Application Data
(60) Provisional application No. 63/472,072, filed on Jun. 9, 2023.

(51) Int. Cl. G16H 50/20; G01N 15/10; G01N 15/12; G16B 40/10; G16B 40/20

(52) U.S. Cl. CPC G16H 50/20; G01N 15/12; G16B 40/10; G16B 40/20; G01N 2015/1006

(57) ABSTRACT

The disclosure includes a platform for classifying particles (e.g., biological cells) based on impedance measurements of individual particles using a biodevice. A machine learning model can be used to classify cells with high accuracy (e.g., greater than 95%). This can be accomplished without the need for sample preparation or cellular tagging. The biodevice can immobilize individual cells (e.g., mechanically immobilize with a micro-pore). An external AC waveform can be applied, for example, in the 1 Hz to 1 MHz range. The machine learning model may be generated to provide classifications based on, for example, measurements of impedance magnitude and phase angle at discrete frequencies.

### CROSS-REFERENCE TO RELATED APPLICATIONS

This application claims priority to Provisional Patent Application No. 63/472,072 filed Jun. 9, 2023, the entirety of which is incorporated herein by reference.

### STATEMENT REGARDING FEDERAL FUNDING

This invention was made with government support under 1648035 awarded by the National Science Foundation, and 5R25GM127191-04 awarded by the National Institutes of Health. The government has certain rights in the invention.

### BACKGROUND

In the medical field, there is often a need to identify unknown biological samples to understand underlying symptoms, propose potential treatment methods, and provide medical prognoses. This type of process typically involves a biopsy followed by tissue identification based on the types of biological cells found in the sample. The methods currently employed are based on optical microscopy and flow cytometry. In both cases, considerable sample preparation and fluorescent tagging is required. In addition, access to highly specialized equipment and costly chemical reagents is needed. These methods, however, are only suited to be used in a certified laboratory setting. It would be valuable for individuals to be able to access this kind of information to identify potential illness at earlier stages, analogous to a tool to measure blood glucose levels (e.g., at home).

### SUMMARY

In various embodiments, this disclosure relates generally to, for example, an apparatus that can classify particles based on impedance measurements. The impedance measurements can correspond to individual particles that exhibit an electrical response to, for example, an AC waveform. The particles can, for example, comprise biological cells, but are not limited to biological cells. In some embodiments, the particles may be non-living particles. The disclosure also relates to use of machine learning to classify particles. A machine learning model can be employed to classify or otherwise determine a characteristic of individual particles. The machine learning model may receive impedance data and provide a classification that is indicative of a property (e.g., type of particle, presence of a molecule or other component, etc.). In various embodiments, impedance data can comprise, or be based on, impedance magnitude and phase angle or the real and imaginary components of the impedance at discrete frequencies. It is noted that impedance magnitude and phase angle represent the complex impedance, which has a real component and an imaginary (e.g., a multiple of i or j) component.

In various embodiments, classifying particles can be accomplished without needing sample preparation or cellular tagging. In various embodiments, an apparatus can immobilize individual particles, such as through mechanical immobilization using a micro-pore. In various embodiments, an external AC waveform can be introduced at frequencies ranging from, for example, 1 Hz to 1 MHz range.

These and other aspects and implementations are further discussed in the non-limiting examples provided below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which can be carried on appropriate carrier media (computer readable media), which can be tangible carrier media (e.g. disks) or intangible carrier media (e.g. communications signals). Aspects can also be implemented using suitable apparatus, which can take the form of programmable computers running computer programs arranged to implement the aspect. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 is an example system comprising components capable of implementing various illustrative embodiments of the disclosure.

FIG. 2 is a cross-sectional schematic representation of a biodevice according to various illustrative embodiments of the disclosure.

FIG. 3 depicts equivalent circuits corresponding to an example biodevice such as the biodevice depicted in FIG. 2, according to various illustrative embodiments of the disclosure.

FIG. 4 depicts, at the top, an exploded view of an example biodevice, and at the bottom, an example biodevice mounted on microscope in an experiment, according to various illustrative embodiments of the disclosure.

FIG. 5 depicts real-time monitoring screens displaying the EIS, image of trapped cell, and monitoring of pressure, according to various illustrative embodiments of the disclosure.

FIG. 6 depicts different possibilities of a cell trapped on an example biodevice, according to various illustrative embodiments of the disclosure.

FIG. 7 provides impedance magnitude (Z) vs phase Angle (Ø) plots according to various illustrative embodiments of the disclosure.

FIG. 8A provides mixed data (circles) showing two populations: one close to T-cells (squares), while the other is not (suspects CAR-Ts), according to various illustrative embodiments of the disclosure. FIG. 8B shows magnetically-separated suspects CAR-Ts (triangle) and T-cells form different clusters, according to various illustrative embodiments of the disclosure. FIG. 8C shows suspected CAR-Ts are separated into two clusters, illustrating system sensitivity to distinguish between CAR-Ts, according to various illustrative embodiments of the disclosure.

FIG. 9A shows T-cells and CAR-Ts and separated into different clusters, according to various illustrative embodiments of the disclosure. FIG. 9B shows two clusters are formed from the mixed data, which can be further used as a prediction, according to various illustrative embodiments of the disclosure. FIG. 9C shows the approach identifies CAR-Ts and T-cells from the mixed data, according to various illustrative embodiments of the disclosure.

FIG. 10 shows impedance magnitude Loadings Line Plot in the PC1 for the 12 chips at 90 mV rms, according to various illustrative embodiments of the disclosure.

FIG. 11 shows impedance magnitude Loadings Line Plot in the PC2 for the 12 chips at 90 mV rms, according to various illustrative embodiments of the disclosure.

FIG. 12 shows impedance magnitude Scores Plot at 90 mV rms for the 12 chips (reduced frequency range), according to various illustrative embodiments of the disclosure.

FIG. 13 shows phase angle Loadings Line Plot in the PC1 for the 12 chips at 90 mV rms, according to various illustrative embodiments of the disclosure.

FIG. 14 shows phase angle Loadings Line Plot in the PC2 for the 12 chips at 90 mV rms, according to various illustrative embodiments of the disclosure.

FIG. 15 shows HeLa Impedance Component vs Frequency with Highlighted Outliers, according to various illustrative embodiments of the disclosure. Plots in rows 1-4 represent 30 mV, 60 mV, 90 mV, and 150 mV, respectively. No outliers detected at 120 mV.

FIG. 16 shows MCF Impedance Component vs Frequency with Highlighted Outliers, according to various illustrative embodiments of the disclosure. Plots in rows 1-4 represent 30 mV, 60 mV, 90 mV, and 120 mV, respectively. No outliers detected at 150 mV.

FIG. 17 shows MDA Impedance Component vs Frequency with Highlighted Outliers, according to various illustrative embodiments of the disclosure. Plots in rows 1-4 represent 60 mV, 90 mV, 120 mV, 150 mV, respectively. No outliers detected at 30 mV.

FIG. 18 shows HeLa Impedance Component vs Frequency after outlier screening, according to various illustrative embodiments of the disclosure. Plots in rows 1-5 represent 30 mV, 60 mV, 90 mV, 120 mV, and 150 mV, respectively.

FIG. 19 shows MCF Impedance Component vs Frequency after outlier screening, according to various illustrative embodiments of the disclosure. Plots in rows 1-5 represent 30 mV, 60 mV, 90 mV, 120 mV, and 150 mV, respectively.

FIG. 20 shows MDA Impedance Component vs Frequency after outlier screening, according to various illustrative embodiments of the disclosure. Plots in rows 1-5 represent 30 mV, 60 mV, 90 mV, 120 mV, and 150 mV, respectively.

FIG. 21 shows HeLa-MCF PCA Scores Plot. Letters A-E indicate 30 mV, 60 mV, 90 mV, 120 mV, and 150 mV, according to various illustrative embodiments of the disclosure. Impedance magnitude is denoted by 1 and phase angle by 2.

FIG. 22 shows HeLa-MDA PCA Scores Plot. Letters A-E indicate 30 mV, 60 mV, 90 mV, 120 mV, and 150 mV, according to various illustrative embodiments of the disclosure. Impedance magnitude is denoted by 1 and phase angle by 2.

FIG. 23 shows MCF-MDA PCA Scores Plot, according to various illustrative embodiments of the disclosure. Letters A-E indicate 30 mV, 60 mV, 90 mV, 120 mV, and 150 mV. Impedance magnitude is denoted by 1 and phase angle by 2.

FIG. 24 shows phase angle Loadings Line Plot in the PC1 for HeLa and MCF12A at 90 mV rms, according to various illustrative embodiments of the disclosure.

FIG. 25 shows phase angle Loadings Line Plot in the PC2 for HeLa and MCF12A at 90 mV rms, according to various illustrative embodiments of the disclosure.

FIG. 26 shows phase angle Scores Plot at 90 mV rms for HeLa and MCF12A, according to various illustrative embodiments of the disclosure.

FIG. 27 shows impedance magnitude Scores Plot at 90 mV rms for HeLa and MCF12A, according to various illustrative embodiments of the disclosure.

FIG. 28 shows phase angle Scores Plot at 90 mV rms for MDA-MB-231 (MDA) and MCF12A (MCF), according to various illustrative embodiments of the disclosure.

FIG. 29 shows impedance magnitude Scores Plot at 90 mV rms for MDA-MB-231 and MCF12A, according to various illustrative embodiments of the disclosure.

FIG. 30 shows phase angle Scores Plot at 90 mV rms for HeLa and MDA-MB-231, according to various illustrative embodiments of the disclosure.

FIG. 31 shows impedance magnitude Scores Plot at 90 mV rms for HeLa and MDA-MB-231, according to various illustrative embodiments of the disclosure.

### DETAILED DESCRIPTION

Identifying unknown components of biological or other samples can provide a better understanding of, for example, patient symptoms, treatment options, and prognoses. Diseases (such as, but not limited to, cancer) have a much better prognosis when detected at earlier stages. In example embodiments, an apparatus classifies particles to, for example, identify potential illnesses such as cancer at earlier stages.

To help with adoption by patients and health care providers, a system for biological cell identification and/or classification that is relatively easy to use, with a robust setup, is provided, in example embodiments. An apparatus can be used to classify biological cells and/or other particles based on impedance measurements of the individual cells and/or other particles (e.g., impedance magnitude and phase angle). A machine learning model (e.g., employing random forest or other machine learning architectures) can be used to classify cells with greater accuracy (e.g., at least 85%, at least 90%, or at least 95% accuracy).

In various embodiments, the apparatus can immobilize individual cells, such as through mechanical immobilization using a micro-scale pore. An external excitation waveform can be introduced, using, for example, non-polarizable electrodes. Example non-polarizable electrodes include Ag/AgCl electrodes and platinum black. The apparatus may be housed in a polycarbonate manifold.

The electrical waveform may be controlled by, for example, a potentiostat which uses electrochemical impedance spectroscopy (EIS) to record the impedance of each cell and/or other particle at discrete frequencies in the, for example, 1 Hz to 1 MHz range. A random forest architecture, for example, may be used to provide a model for cellular (or other particles) classification and identifies the most relevant frequencies that enable the classification. In some embodiments, the electrical recording platform has a simplicity analogous to a blood glucose monitor found at pharmacies, clinics, or other locations. In some embodiments, the machine learning model could be incorporated by uploading a relatively small-sized file to a cloud server via a client application (e.g., a smartphone or other mobile computing device application or “app”). This technology can be used to identify, for example, bloodborne diseases in blood biopsies (e.g., lymphoma, leukemia) and/or to provide surrogate potency assays for the manufacturing of cell-based therapies (e.g., CAR T cells).

A different machine learning model may be generated for each set of particles (e.g., each pair of particles, or each of three or more particles) to be distinguished from each other. For example, one model may be generated for cancerous/healthy cell classification, another for T-cell classification, another for other disease/healthy classification, and yet other models may be generated for classification of a cell or other particle into one of three particular categories or classifications, and still other models for classification of a cell or other particle into one of more than three particular categories or classifications.

Different models may have different parameters. For example, different random forest models may have different numbers of trees/nodes depending on the different datasets used to train the models. If random forest architectures are employed, generally, as the number of trees/nodes is increased, the efficiency of classification by the model can reach a plateau. Thus, for each random forest machine learning model, the number of trees/nodes that maximizes the classification efficiency can be based on where the models plateau. Other machine learning architectures may be employed as well, with analogous model inputs and outputs.

Although T-cells are discussed as example use cases, various embodiments can relate to other use cases, such as, for example without limitation, HeLa cells (cervical cancer cells), MDA-MB 231 (breast metastatic cancer cells), MCF12a (healthy breast cells), Jurkat cells (immortalized human T lymphocyte), CD4+T-cells, CD3+T-cells, CAR T-cells (created from CD3+T-cells), and/or other cells.

Experiments have been performed with CAR T-cells that have been positively selected via MACS® Cell Separation technology (Miltenyi Biotec). MACS® (“MAgnetic Cell Separation”) technology uses magnetic nanoparticles to label the target cell to be enriched and separates them by using a proprietary magnetic column (https://www.miltenyibiotec.com/US-en/products/macs-cell-separation.html). Since the cells are positively selected, they end up with a coating of magnetic nanoparticles in the surface. The biological activity of cells separated by this technology is not affected. CAR T-cells can be separated from a mixed batch of T-cells and CAR Ts in this way. These magnetically separated CAR Ts can be characterized and the results compared with the characterization of a mixed batch of T-cells and CAR Ts and the characterization of just T-cells (same used to make the CAR Ts).

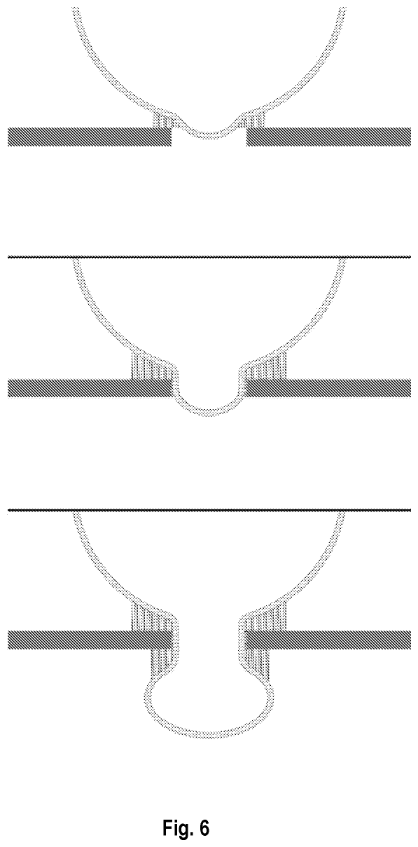

In an example, principal component analysis (PCA) revealed four distinct groups (see FIG. 6): (1) T-cells depicted as squares, (2) the mixed batch in the leftmost oval depicted as circles, (3) the mixed batch in the rightmost oval depicted as circles, and (4) magnetically separated CAR Ts depicted as triangles. The mixed batch circles that are found in the leftmost oval may be T-cells, because the data correlates well with the T-cell data. The circles that are found in the rightmost oval may be CAR Ts. The system can be sensitive enough to distinguish between CAR Ts and magnetically separated CAR Ts. Accordingly, cells labelled with magnetic nanobeads can be distinguished from non-labeled cells.

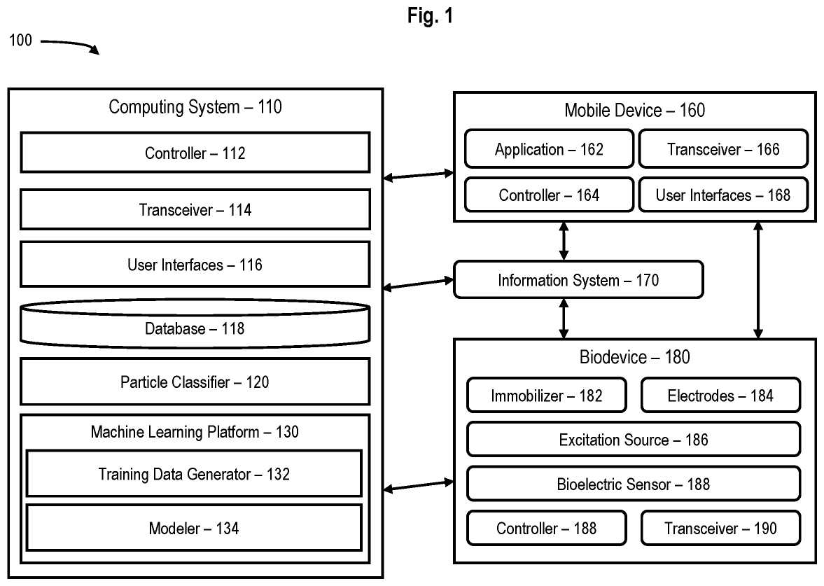

Referring to FIG. 1, in various embodiments, a system 100 may include a computing system 110, a mobile device 160, an information system 170, and a biodevice 180. The computing system 110 may be, or may include, one or more computing devices, co-located or remote to each other. The mobile device 160 may be, for example, a smartphone, tablet, or another computing device capable of provided various generic and/or specialized functionality. Information system 170 may be, or may include, a health information system and/or a management information system that may record and/or provide health or other information. The biodevice 180 may be capable of obtaining data on samples with cells or other particles to be classified or otherwise characterized, such as materials obtained from human or non-human subjects, or derived from such materials. System 100 thus includes hardware and software capable of implementing various embodiments of the disclosed approach.

The computing system 110 (e.g., one or more computing devices) may be used to control and/or exchange signals and/or other data with mobile device 160, information system 170, and/or biodevice 180, directly (e.g., through wireless and/or wired communication) or indirectly via another component of system 100 (e.g., via any combination of wireless and/or wired communication). The computing system 110 may include one or more processors and one or more volatile and/or non-volatile memories for storing computing code and data that are captured, acquired, recorded, and/or generated.

The computing system 110 may include a controller 112 comprising one or more processors, one or more memory modules, and firmware or other code executable storable on the one or more memory modules and executable by the one or more processors. Controller 112 may be configured to control components of computing system 110. Controller 112 may also be configured to exchange control signals with mobile device 160, information system 170, biodevice 180, and/or any components thereof, allowing the computing system 110 to be used to acquire and/or control, for example, acquisition of bioelectric signals by sensors or other detectors, positioning or repositioning of samples or devices, recording or obtaining other information, etc.

A transceiver 114 allows the computing system 110 to exchange readings, control commands, and/or other data or signals, wirelessly or via wires, directly or indirectly via networking protocols, with, for example, mobile device 160, information system 170, and/or biodevice 180, or components thereof. One or more user interfaces 116 allow the computing system 110 to receive user inputs (e.g., via keyboard, touchscreen, microphone, camera, motion detection, biometric scan, etc.) and provide outputs (e.g., via display screens, audio speakers, light emitters, AR/VR/MR headsets etc.) with users. The computing system 110 may additionally include one or more databases 118 for storing, for example, data acquired from one or more systems or devices, signals acquired via one or more sensors, images, biomarker signatures, etc. In some implementations, database 118 (or portions thereof) may alternatively or additionally be part of another computing device that is co-located or remote (e.g., via “cloud computing”) and in communication with computing system 110, mobile device 160, information system 170, and/or biodevice 180 or components thereof.

Mobile device 160 may include one or more client applications 162, which may be or may include, for example, any software application that is executed on or by mobile device 160, such as an application for accessing, controlling (e.g., initiating or stopping a test), or acquiring data from biodevice 180. Client application 162 may, for example, collect, preprocess, and/or process bioelectric data from biodevice 180, and generate and/or transmit a computer file containing raw or processed data to computing system 110. The computer file may also include additional information, such as an identification of the biodevice 180, an indicator of the subject from whom a sample was obtained, information on the sample being tested, the test being performed, the types of particles, and/or any combination thereof. Data may be encrypted, and/or or other security measures employed, to maintain the confidentiality and privacy of the information and the integrity of the data.

Mobile device 160 may also include a controller 164 comprising one or more processors, one or more memory modules, and firmware or other code executable storable on the one or more memory modules and executable by the one or more processors. Controller 164 may be configured to control components of mobile device 160. Controller 164 may also be configured to exchange signals and/or data with computing system, 110, information system 170, and/or biodevice 180, and/or any components thereof, allowing the mobile device 160 to be used to acquire and/or control, for example, acquisition of bioelectric signals by sensors or other detectors, positioning or repositioning of samples or devices, recording or obtaining other information, etc. A transceiver 166 allows the mobile device 160 to exchange readings, control commands, and/or other data or signals, wirelessly or via wires, directly or indirectly via networking protocols, with, for example, computing system 110, information system 170, and/or biodevice 180, or components thereof. One or more user interfaces 168 allow the mobile device 160 to receive user inputs (e.g., via keyboard, touchscreen, microphone, camera, motion detection, biometric scan, etc.) and provide outputs (e.g., via display screens, audio speakers, light emitters, AR/VR/MR headsets etc.) with users.

Biodevice 180 may be configured to receive and acquire data on samples containing cells or other particles having two or more potential classifications (e.g., healthy and not healthy). The biodevice 180 may be or may include a microfluidics-based device, with a combination of microfluidics components such as any suitable combination of actuators, chambers, channels, electrodes, injectors, inputs, needles, outputs, pumps, reservoirs, active or passive thermal management systems (e.g., heaters and/or heat sinks), valves, wells, etc. Biodevice 180 may include, for example, an immobilizer 182, such as a mechanical immobilizer (e.g., a micropore trap) that limits the movement of one or more particles to be tested. An excitation source 186 may be used to apply, to an immobilized particle, waveforms via electrodes 184. A bioelectric sensor 188 may be used to detect various electrical signals (e.g., impedance) that is to be analyzed to make determinations regarding one or more particles being tested. Biodevice 180 may incorporate or function in conjunction with, for example, a potentiostat and electrochemical impedance spectroscopy (EIS) components.

Particle analyzer 120 may retrieve or otherwise receive data (e.g., from or via biodevice 180) and analyze or otherwise process the data to determine a classification or characteristic of a particle based on the data. The particle analyzer 120 may employ one or more machine learning models. In some embodiments, the particle analyzer selects which model to apply based on information on which test was performed or other information (e.g., information received from the mobile device 160, the information system 170, and/or the biodevice 180.

Machine learning platform 130 may be configured to generate, train, and update machine learning models, as further discussed herein. Machine learning platform 130 may, for example, employ certain machine learning techniques and algorithms to train and update predictive models. Machine learning platform 130 may include a training data generator 132 which may, for example, generate or otherwise obtain training data, such as impedance and phase angle data and/or labels that identify a characteristic of components of particles corresponding to the data. The modeler 134 may use training data to generate, train, and/or update models that may then be used, for example, for particle classification by particle classifier 120.

The system 100 need not have all of the components in FIG. 1, and similarly, system 100 is not limited to only the components in FIG. 1. Similarly, the computing system 110, the mobile device 160, and/or the biodevice 180 need not have all the components illustrated in FIG. 1, nor are any of them limited to having only the components depicted in FIG. 1. Moreover, various elements of FIG. 1 may be rearranged or reorganized such that certain functionality may be provided by different components and/or by multiple components. FIG. 1 thus is illustrative of various embodiments, but is not all-inclusive of all potential embodiments. In various implementations, components of system 100 may be rearranged, integrated, or split up in other configurations. For example, computing system 110 (or components thereof) may be integrated with one or more of the mobile device 160, information system 170, biodevice 180, and/or components thereof. Not all components of system 100 depicted in FIG. 1 are required to implement the disclosed approach, and in various embodiments, only a subset of the components of system 100 may be employed. For example, in various embodiments, computing system 110 may obtain and process data that was obtained via an another system that is, or is not, in direct communication with the computing system 110. Similarly, one or more of computing system 110, mobile device 160, information system 170, and/or biodevice 180 may process certain data.

### Examples

A. Impedance-Based CAR T Cell Identification after T-Cell Transduction: A Label-Free Tool in Cell Manufacturing Technologies

For the allogenic Chimeric Antigen Receptor (CAR) T-cell immunotherapy to become scalable, the potency of thawed CAR T-cells from stock should be quantified. The goal of this example embodiment is to correlate CAR T-cells biological potency through electric measurements. The first step is to differentiate between T-cell and CAR-expressing T-cells. Various embodiments provide systems and methods for such differentiation.

Example embodiments use electrochemical impedance spectroscopy (EIS) to obtain electrical data non-invasive, label-freely. EIS is a technique that measures the impedance of a system as a function of the AC potentials for a given frequency. In various embodiments, EIS bioelectrical measurements are distinct between T-cells and CAR T-cells, and EIS is sensitive enough to distinguish electrically between active and inactive CAR T-cells. In an example fluidic device, a single cell is trapped a silicon micropore chip where silver electrodes are used to measure the cell's bioelectrical response. In example embodiments, EIS was performed using two AC voltages (30 mV and 45 mV).

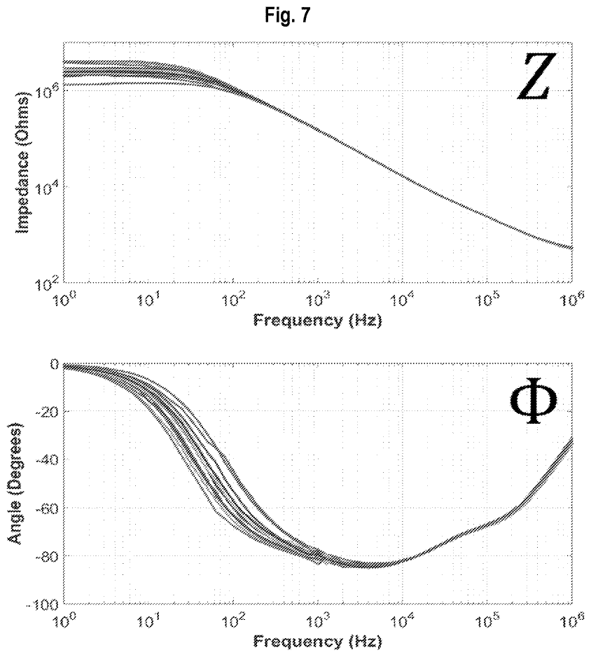

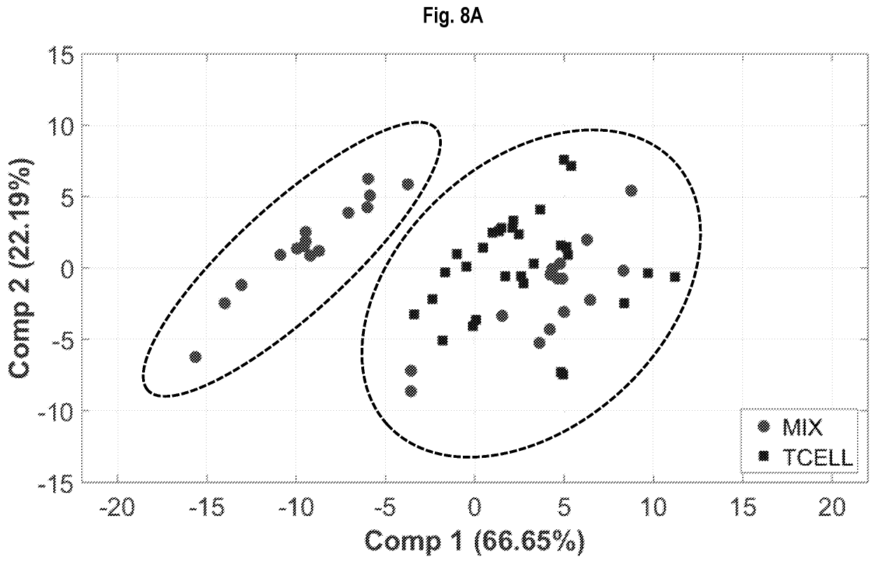

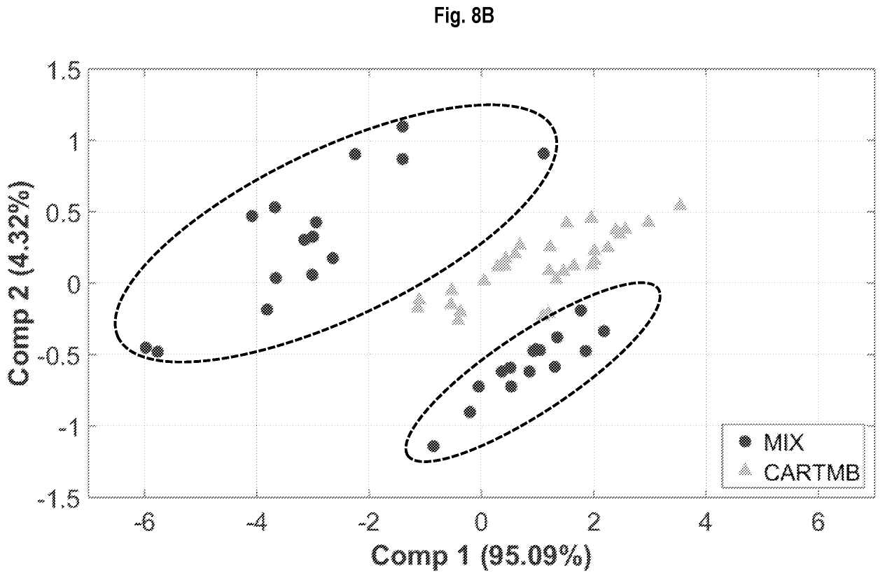

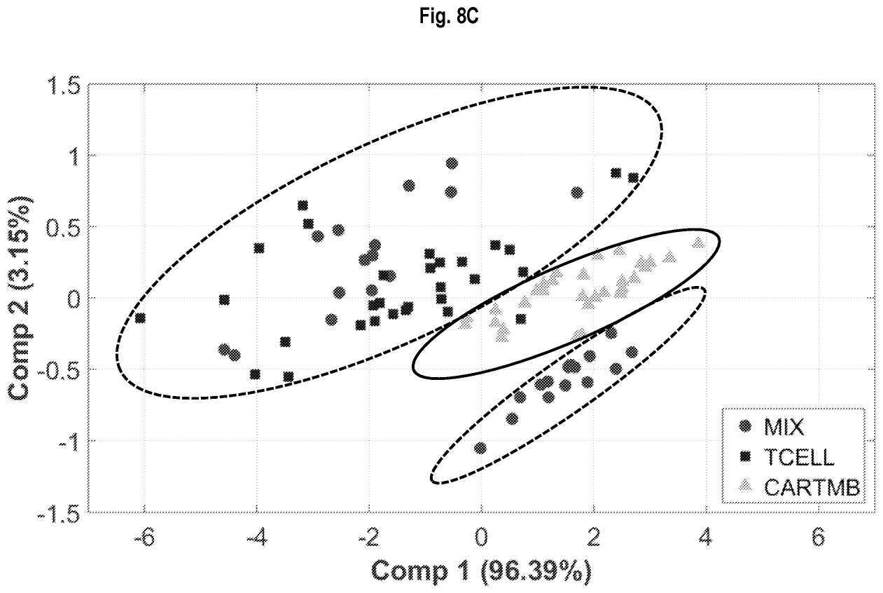

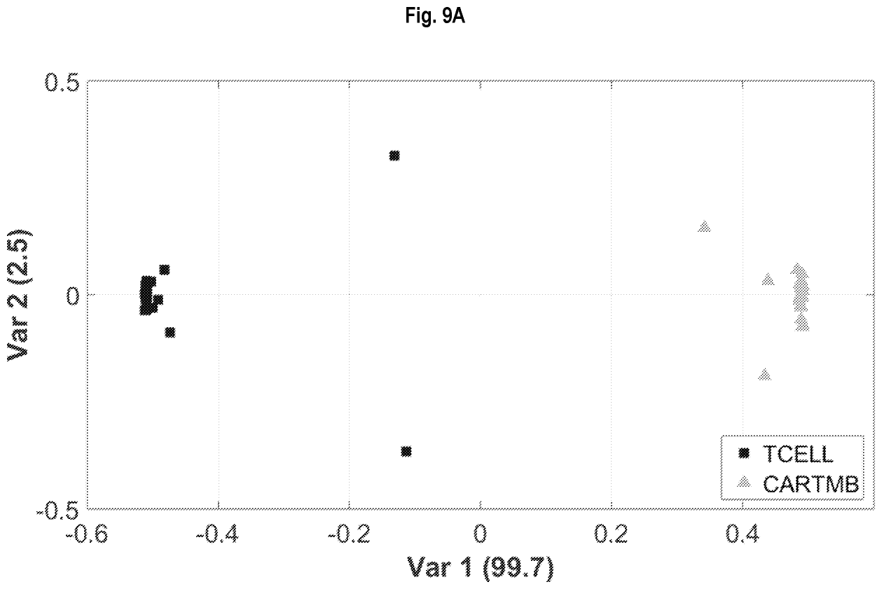

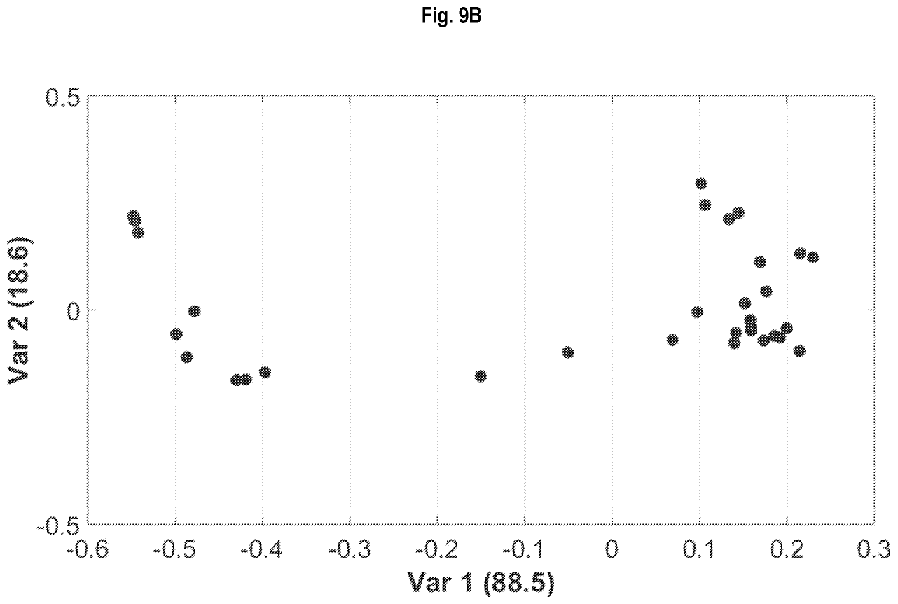

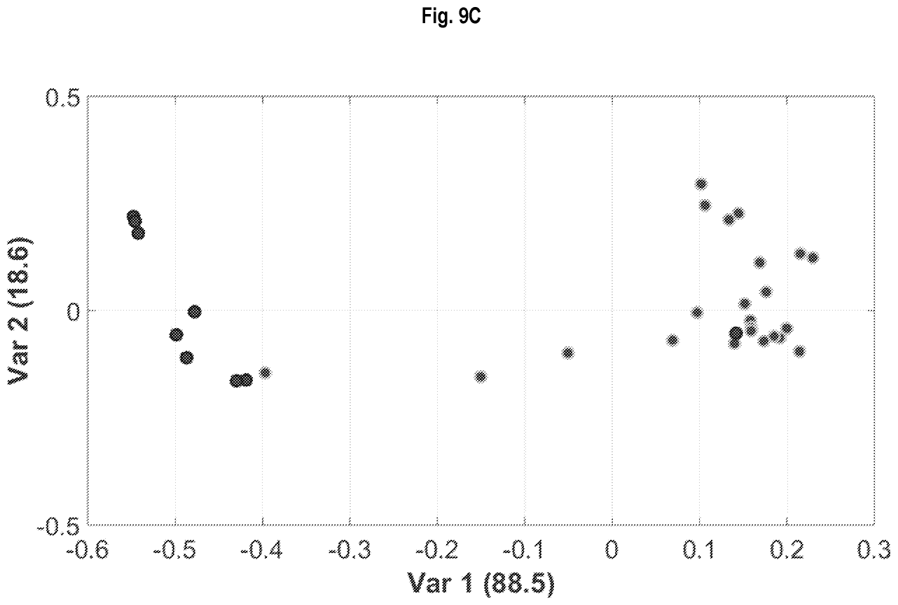

Various embodiments employ machine learning algorithms for analysis to find differences between cell populations. Two methods were tested, where clustering means similarity between data points: principal component analysis (PCA), for which data is un-labeled; and random forest, for which data is labeled. Three experimental cell groups were tested. Group A: T-cells+Transduced CAR T-cells. Group B: CAR T-cells Magnetic Beads. And Group C: T-cells. Impedance Magnitude (Z) and Phase Angle (@) were obtained as a function of the AC frequency ranging from 1 Hz to 1 MHz, plots of which are shown in FIG. 7. Data based on PCA analyses are plotted in FIGS. 8A, 8B, and 8C, and data based on random forest modeling are plotted in FIGS. 9A, 9B, and 9C.

### B. Electrochemical Impedance Spectroscopy Characterization and Identification of Cancer Cells

In biomedical sciences and engineering, microfluidic systems can be used for the analysis of cellular structures at the micro and nanoscales to identify disorders and mutations that can lead to cancer. In the metastatic process, cancer cells break away from the initial tumor site, travel through the blood or lymph system, and form new tumors by colonizing distal sites. Even though this process begins in the early stages of cancer progression, experimental studies have shown that approximately 0.02% of cancer cells injected into the circulation form metastatic foci. The inefficiency of the metastatic process provides a window of opportunity to detect cancer in the early stages by identifying such tumor cells in the bloodstream. Early detection of cancer can be crucial for the survival rates of patients. However, most current diagnostic tools are only capable of detecting cancer during its terminal stages, at which point patients have faced a staggering 90% mortality rate due to metastasis or high stage cancers. However, the task has proven to be difficult due to low tumor cell count and inconclusive surface markers for target cell detection.

Example embodiments provide a different approach towards the advancement of cancer cell research by integrating microfluidics with electrical recordings at the single cell level, employing electrochemical impedance spectroscopy (EIS). By performing a frequency sweep and recording the electrical response of the cancer cell lines under study, surface and volumetric characteristics of interest can be interpreted. Results show different electrical behavior in certain frequency ranges among the studied cell lines that comprise the highest quality of differentiation between the discussed biological cells. This technology can potentially be used to detect anomalous/cancerous cells in blood biopsies, which will be an important step in the early detection of diseases such as cancer.

Research shows that early cancer can be detected by identifying circulating tumor cells (CTCs) and cancer stem cells (CSCs). CTCs are cancer phenotype cells responsible for cancer distal colonization located in peripheral blood, while CSCs promote cancer tumor formation and maintenance, growth, and resistance mechanisms against radiation and chemotherapy. Due to their presence in peripheral blood, low cell count in the bloodstream, and unknown cancer cell vulnerabilities, the separation and characterization of CTCs and CSCs can provide a useful surrogate with which cancer status can be evaluated through a non-invasive procedure.

However, the detection of CTCs and CSCs is challenging. Some prior methods are distinguished for high sensitivity and antibody detection, cell quantification through morphological cell analysis, and tumor specific protein assay. But these prior methods of detecting CTCs and CSCs still do not offer precise identification. The disclosed approach to identifying these cells with more certainty can be crucial.

Example embodiments may employ electrochemical impedance spectroscopy (EIS), which can be non-invasive and label free. EIS can be used to measure the impedance across an electrochemical cell by applying an external AC voltage in a specified frequency range. Performing a frequency sweep and recording the electrical response of subjects of interest allows one to elucidate surface and volumetric characteristics of interest. EIS can be employed in identifying biological cell types by characterizing their unique bioelectric spectra, an approach that is applicable to, for example, the identification of cancer cells.

Most research in the study of the electrophysiological properties of cells has focused on measuring the electric properties of the cellular membrane. Example embodiments may employ a microfluidic system to capture and electrically characterize individual cells or other particles based on, for example, impedance measurements. This device allows researchers to observe and understand the cell as a whole organism, aiding characterization of cells as abnormal or normal. Example systems can have several functions and advantages, such as, smaller dimensions, lower costs due to batch fabrication, incorporation of sensing, signal conditioning, superior functionality, and the ability to provide redundancy and enhanced reliability. Example embodiments provide a noninvasive, label free, low cost, and reliable system for disease detection, such as early cancer detection.

As discussed, cancer detection at an early stage raises the possibility of survival. The disclosed approach provides a robust scheme of early detection of diseases (such as, but not limited to, cancer). Example embodiments consider that each type of cell has a unique electric signature that can be recorded by the application of an externally applied electric field. Embodiments of the disclosed approach can be used to characterize cells (e.g., cancer cell lines) through EIS. In example embodiments, a micropore trap device is used to capture one biological cell through application of a pressure differential. Once immobilized, the biological cell's electric spectra can be recorded through EIS. In various embodiments, bioelectric characterization of cell lines of interest is based on the impedance and phase angle measurements as a function of frequency.

In example experiments, a complete bioelectrical signature for Hela, MB-MDA-231 and MCF12A was conducted. Electrical recordings were performed in five different excitation voltages in the 1 Hz to 300 KHz frequency range. The experiment demonstrated a high level of differentiation between pairs of cell lines using principal components analysis (PCA) and Random Forest (RF). This approach be used by the scientific and medical community by, for example, characterizing cell lines, thus distinguishing between healthy and cancerous peripheral cells, including CTCs and CSCs.

### B1. Cell Preparation and Experimental Setup

Hela, MDA-MB-231 (American Type Culture Collection, USA) cancer cells, and human breast tissue cell line MCF12A (American Type Culture Collection, USA) were used in this experiment. Cancerous cells were cultured with 90% DMEM media, 2 mmol/L L-glutamine, 10% preheated fetal bovine serum, 100 IU/mL penicillin. Human breast cells were cultured with DMEM/F12 media, 2 mmol/L L-glutamine, 10% preheated fetal bovine serum, 100 IU/mL penicillin, epidermal growth factor, 100 μg/mL streptomycin, 10 μg/mL bovine insulin, and 100 ng/mL cholera toxin. For subculturing, cells were maintained in a 5% CO₂ incubator at 37° C.; every 2 to 3 days, the culture media was changed. For experimentation, cells were subcultured 48 hours prior to use. A hemacytometer and trypan blue were used to test for confluence. Confluence must be above 90% and viability above 90% for a cell batch to be considered viable. Prior to experimentation, cells were removed from the T25 flask with trypsin-EDTA (Fisher Scientific, USA). Suspension cells were then centrifuged at 3500 RPM at 4° C. for 5 minutes. The supernatant was removed, and the cell pellet resuspended with phosphate buffer saline (PBS) 1X up to a density of 1 million cells mL⁻¹.

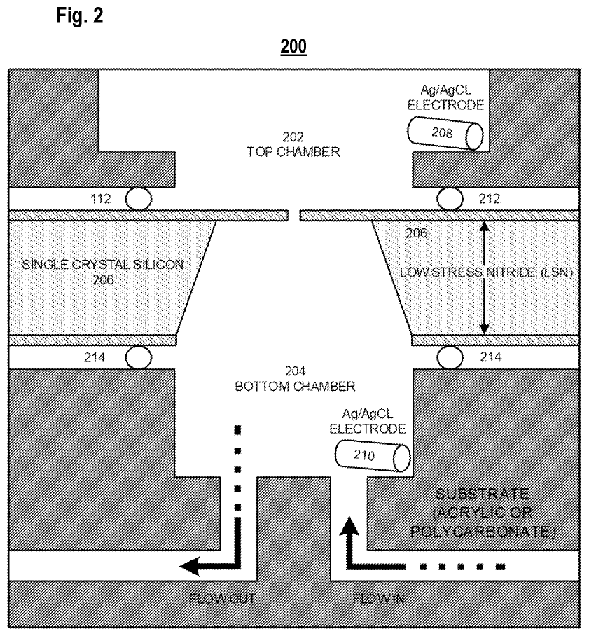

Individual cell isolation was achieved using a device designed and manufactured to obtain electric cell properties. An example embodiment of such a biodevice 200 is shown in FIG. 2. Biodevice 200 includes two polycarbonate chambers (labeled “top chamber” 102 and “bottom chamber” 104) divided by a dielectric silicon chip 106. The top chamber 102 has an Ag/AgCl electrode 108 (Warner Instruments, USA) and cells suspended in PBS. The bottom chamber 104 has an Ag/AgCl electrode 110 and two fluid exits, where one creates negative pressure and the other captures a cell in a micropore. The silicon chip 106 contains the micropore, with a diameter of 3 μm. To avoid fluid leaks, an O-ring 112 (Apple Rubber, USA) is installed between the top chamber and silicon chip 106. The same process was used by installing O-ring 114 between the bottom chamber 104 and silicon chip 106. The device 100, its two chambers, and the fluidic entry and exit, were filled with PBS. A pressure sensor was installed at the fluid exit with a T-connector to record system pressure. The fluid entry channel contains a syringe with PBS to control the system's pressure. Pressure sensing data was obtained using Lab View (National Instruments Corp, USA). This device was mounted on a microscope (Olympus LS, Japan) within a Faraday cage to avoid electric noise. Finally, the potentiostat (Gamry Instruments Inc, USA) was connected to both Ag/AgCl electrodes 108 and 110.

### B2. Device Calibration

Ag/AgCl electrodes could have a different potential, affecting the pseudo-linearity response on EIS measurements. This differentiation can be solved by balancing electrodes' potential. Potential balance was processed using the Ag/AgCl electrodes protocol according to manufacturer.

### B3. Equivalent Circuit

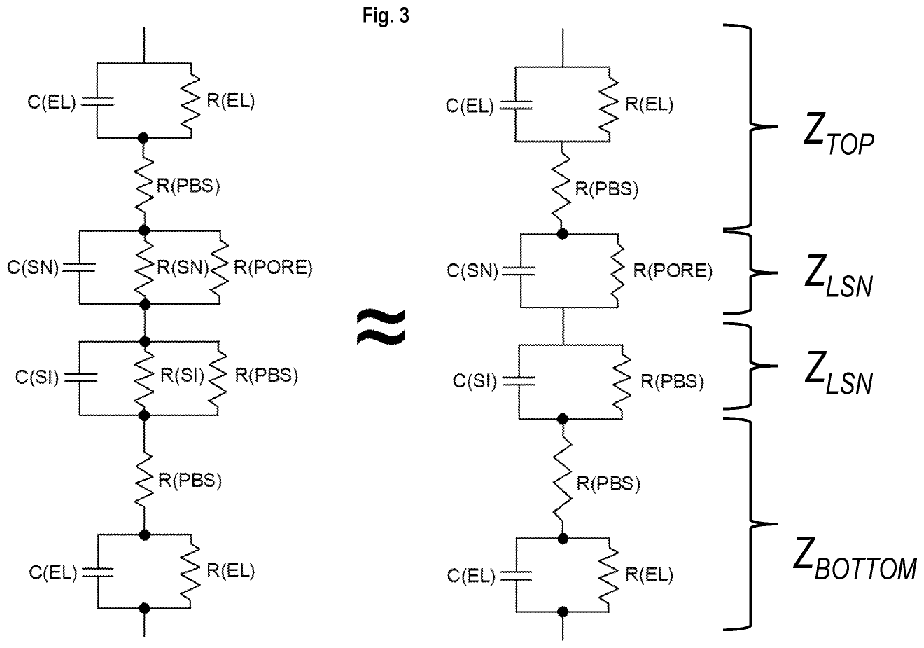

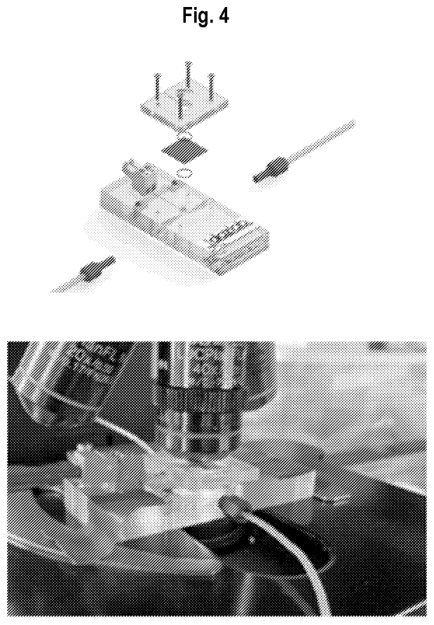

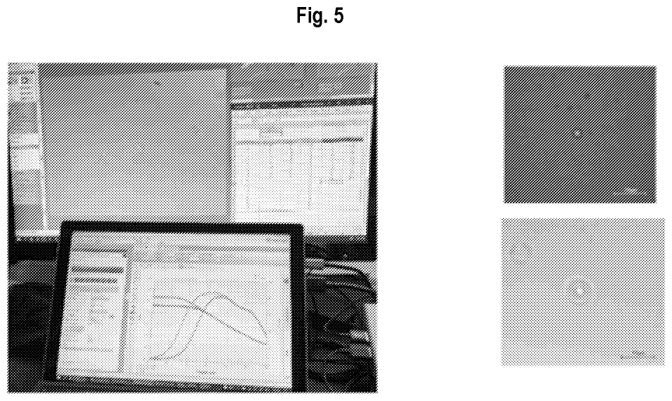

Biological cells are complex electric circuits in nature. In order to understand their electric behavior, different equivalent circuit models were created. Most of the circuits emulate the cell as dielectric particles that are polarized in an electric field. This approach evaluates the cell as a particle that contains a lot of mini charges inside. Hence, electronic circuit cell representation simplifies the complex cell configuration. Discrete electric equivalent circuit for the microfluidic system was created. Two main configurations were considered during experimentation. The first configuration is the device without a captured cell. The representation includes a top electrode acting as a resistor-capacitor (RC) circuit, a phosphate-buffered saline (PBS) in the top chamber working as a resistor, a low-stress nitride (LSN) acting as an RC circuit, a micro-pore resistor, a silicon chip acting as an RC circuit in parallel to a resistor by PBS, and finally, a bottom electrode similar to the top electrode. The second configuration included the microfluidic device and the cell trapped in the micropore. Equivalent circuits are shown in FIG. 3.

Equivalent circuits present an opportunity to streamline operations, where the first circuit serves as a baseline and the second circuit serves the purpose of cell-baseline measurement. By considering this approach, a straightforward mathematical subtraction can yield the following equation.

$${Z}_{Cell - Pore} = {[ \frac{1}{{R}_{Leak}} + {Z}_{Cell} ]}^{- 1} - {R}_{Pore} \qquad ( 1 )$$

This equation shows the measurement in the micropore. The resistance R_{Leak} is generated by the interaction between the cell membrane and the LSN surface. This resistance is directly proportional to the degree of cell-LSN attachment.

Theoretical estimation of cell impedance on this device is determined by the shape of the cell trapped in the micropore, cell is induced under deformation due to trapping pressure, shape is pressure dependent and could be multiple possible scenarios. FIG. 6 shows the different trapping types of cells, these generated changes in R_{Leak}. The cells can be over posed on the micropore, can be in the perfect way (fit the entire micropore volume), or can be exceedingly under the micropore. These scenarios are determinant to the resistance of the system, if the cell is in contact with the LSN, this creates more resistance on the surface, resulting in more cell impedance and vice versa.

$${Z}_{Cell} = \frac{{R}_{L} ( {R}_{c} {R}_{T} {ω}^{2} {C}^{2} + 1 )}{{R}_{T}^{2} {ω}^{2} {C}^{2} + 1} + \frac{{R}_{L}^{2} ω C}{{R}_{T}^{2} {ω}^{2} {C}^{2} + 1} i \qquad ( 2 )$$

where R_{L} is the resistance leak of the cell and surface contact resistance, R_{c} is the resistance of the cell, R_{T} is the total resistance R_{L}+R_{c}, and C is the capacitance of the cell.

### B4. EIS Measurements

Two different data sets were created. One set consisted of impedance measurements without cells (baseline) and the other with cells. The characterization was performed using five different voltages rms, 30 mV, 60 mV, 90 mV, 120 mV, and 150 mV on a frequency range from 1 Hz to 300 kHz. Before any experiment, each voltage had three baseline runs (only PBS) in the chamber for the first data set followed immediately by five runs with cells per voltage, each with a different trapped cell. Forty tests were applied per cell line per voltage rms. To perform the EIS experiment, electrical data was recorded using a potentiostat (Gamry Instruments Inc, USA) and the Gamry Framework program.

When the biodevice was ready with the cells inserted, a cell was captured with a pressure variation using the syringe. Cells' morphology, pressure, room temperature and room humidity were monitored prior and post testing.

### B5. Statistical Analysis

Through MATLAB, EIS output data was screened for outliers, and merged into data sets according to impedance response, AC voltage and cell lines compared (see Supplementary Information). In R, PCA Scores Plots were then used to visualize EIS response clustering in terms of cell line, and in validating device calibration. On the other hand, RF pairwise classification models between HeLa, MDA, and MCF cell lines using their recorded bioelectric spectra were built in R, as well as a classification model for the three. The total number of trees and number of variables considered per tree node were iterated upon finding the values that yield the lowest possible Out of Bag Error Rate (OOBER). The accuracies obtained through the OOBER were then validated with Leave-One-Out Cross Validation (LOOCV).

### B6. Results

Potentiostatic EIS tests were performed on the biological cell lines in the voltages and frequency ranges previously stated. Cell impedance and phase angle were obtained as a function of frequency. Finally, PCA and RF between cancerous and healthy cell lines were conducted.

EIS obtained data represents the magnitude and phase angle as a function of frequency of each cell line. Forty cell samples per voltage (30 mV, 60 mV, 90 mV, 120 mV and 150 mV) and 56 frequencies (10 per decade) were used.

For each PCA statistical analysis, only two sets of cell lines were compared. The first is Hela, a commonly-use cancer cell line, versus MCF, a healthy breast cell line. The second case compares MDA, a cancerous breast cell line versus MCF, a healthy breast cell line (same cell line but cancerous and non-cancerous cells). The data was processed, transformed, and presented in the PC plots using R studio (Posit Software, USA). The influence of variation for each variable (frequencies) can be observed in the loadings line plot (LLP). To visualize the magnitude of the deviation, a scores plot was developed for the impedance magnitude and phase angle, respectively. The data was scaled or standardized by removing the baseline and dividing the data by the standard deviation. A scores plot describes the data structure in terms of sample patterns, showing correlation and variance between samples, as opposed to the loading line plot, which shows variance between variables. In addition, a 99% confidence limit was used for the hoteling ellipses applied to the scores plots, which represents 90% of the explained variance in the data.

For Hela-MCF PCA plots at 60 mV and 90 mV rms, data is stable and below the electroporation point. Raw PCA data do not show an excellent cluster differentiation between cell lines. Frequency ranges were selected for further analysis. These frequency ranges were selected based on the LLP, ensuring that frequencies where the LLP value was under 0.26 were not selected, which means that it was not possible to differentiate one cell line from the other in any frequency. PCA of selected frequencies were constructed.

PCAs using the magnitude of the impedance do not show any clustering of the cell lines. However, phase angle showed a clear clustering between cancerous and non-cancerous cells.

For the MDA-MCF PCA plots, at 60 mV and 90 mVrms, data is stable below the electroporation point. In this case, both voltages lack differentiation with a change in impedance magnitude. Furthermore, the phase angle at 60 mV does not show clear clustering of both cell lines. However, there is a clear differentiation between cancerous and non-cancerous cell lines at 90 mV.

On the other hand, pairwise RF models yielded high accuracies as calculated from OOBER and validated with LOOCV (Table 1). It is noted that classification between HeLa and MDA yielded slightly lower accuracies in the pairwise model. The three-cell line also obtained high accuracy over the tested voltages. Confusion matrices (see Supplemental Information below) show that classification between HeLa and MDA also yielded higher error, consistent with the previous models. It is also worth emphasizing that the model accuracies obtained from OOBER are in close agreement with the accuracies obtained from validation.

TABLE 1
Random Forests Models Generated along with Accuracy Calculated by Out of Bag Error
Rate (OOBER) obtained at model generation and accuracy given by Leave One Out
Cross Validation, for pairwise comparison models and 3- cell line models

| | | Accuracy | | | | | | | |
|---|---|---|---|---|---|---|---|---|---|
| | | | | | | | | HeLa vs MCF | |
| Impedance | Voltage | HeLa vs MCF | | HeLa vs MDA | | MCF vs MDA | | vs MDA | |
| Component | (mV) | LOOCV | OOBER | LOOCV | OOBER | LOOCV | OOBER | LOOCV | OOBER |
| Phase | 30 | 100.0 | 100.0 | 100.0 | 100.0 | 98.7 | 98.7 | 98.2 | 99.1 |
| Angle | 60 | 100.0 | 100.0 | 94.3 | 94.3 | 97.4 | 98.7 | 94.3 | 95.2 |
| | 90 | 100.0 | 100.0 | 95.8 | 97.2 | 100.0 | 100.0 | 97.2 | 97.2 |
| | 120 | 100.0 | 100.0 | 95.8 | 95.8 | 100.0 | 100.0 | 96.3 | 97.2 |
| | 150 | 98.7 | 98.7 | 84.3 | 87.1 | 100.0 | 100.0 | 90.5 | 91.4 |
| Magnitude | 30 | 100.0 | 100.0 | 90.5 | 93.2 | 100.0 | 100.0 | 97.3 | 98.2 |
| | 60 | 100.0 | 100.0 | 94.3 | 94.3 | 100.0 | 100.0 | 97.1 | 97.1 |
| | 90 | 100.0 | 100.0 | 88.9 | 93.1 | 98.6 | 100.0 | 97.2 | 98.2 |
| | 120 | 100.0 | 100.0 | 95.8 | 97.2 | 100.0 | 100.0 | 99.1 | 99.1 |
| | 150 | 100.0 | 100.0 | 90.0 | 90.0 | 98.6 | 100.0 | 94.3 | 94.3 |

Solving the ambiguity of the optimal working voltage for differentiation was crucial for these cell types. In all cases, based on the loading line plots, 60 mV and 90 mV resulted in the best voltages for differentiation. Nevertheless, the most critical case is that of MDA versus MCF12A, where the same cell line was presented, and the only difference was the cancerous mutation on one of the cell lines (MDA). This means that both cell lines contain very similar characteristics. Finally, the optimal working voltage for PCA is at 90 mV.

The PCA results show two behaviors. First, all PCA impedance magnitude results did not differentiate cell lines. This behavior can be attributed to the fact that the device's impedance is more relevant than the cell's impedance in the EIS after 100 Hz. For this reason, baseline and cell impedance are of the same value in some frequencies. Moreover, in low frequencies, the current field travels around the cell; thus, this measure is dependent on the size of the cell and the R_{Leak}. If these two are large, the impedance measurement is high and vice versa.

Furthermore, the smooth lines above indicate that the data is trustworthy, since the behavior is not irrational, and that a trace of a bioelectric fingerprint could be detected. The voltage has been a determining factor in the difference between cell lines since an increase in voltage increased the difference at higher frequencies below electroporation voltage point. However, at low voltages, the differentiation was noticeable at lower and higher frequencies. A stable differentiation can be observed for the selected frequencies in the phase angle for the Hela/MCF, and MDA/MCF cell line comparisons. Furthermore, the phase angle showed the highest level of differentiation for all cell lines, which expressed how much the imaginary component of the impedance is changing with respect to the real component. For cell line comparisons in which frequencies did not show a variance correlation of 0.3 or higher, the theoretical measurements performed with DC voltage differed from the measured values as rms voltage was applied.

In terms of the RF models, highly accurate models were obtained. Results are deemed to be reliable given that the validation accuracy lies in agreement with that obtained at model generation. The HeLa-MDA comparison yielded slightly lower results than the other 2, over the tested voltages and impedance components evaluated. This likely hindered the accuracy of the three cell line models, as also evidenced from the confusion matrices (see Supplemental Information below). Nonetheless, having obtained high accuracy between MCF and MDA classification models shows that using bioelectric properties of cells can allow for differentiation between a healthy and cancerous cell, of the same tissue. Furthermore, in contrast to PCA, RF models provide a method for automatic prediction of an unknown sample's cell line in a highly accurate manner, based on historical data. PCA finds the most important variables for group clustering but does not necessarily provide a practical method for prediction and assessing accuracy. Additionally, like other machine learning models, it has the capability to optimize its performance as new data enters. Thus, the results obtained show that, in various embodiments, coupling machine learning with EIS data enables early, high throughput, and low-cost detection of cancer cells.

High differentiation between two cancerous and a non-cancerous cell lines was observed. The cell lines contained a unique electric pattern or bioelectric fingerprint. This can be used in EIS and RF to identify two cell line groups without the use of biomarkers. In addition, this opens the possibility of using this technique to identify CSCs, CTCs, or any other cell line of interest in the bloodstream. Thus, achieving a label free surrogate technique to identify cancer in its early stages.

In example embodiments, high differentiation between two cancerous and a non-cancerous cell lines was observed. The cell lines contained a unique electric pattern or bioelectric fingerprint. This can be used in EIS and RF to identify two cell line groups without the use of biomarkers. In addition, in other embodiments, this approach can be used to identify CSCs, CTCs, or any other cell line of interest in the bloodstream. Thus, achieving a label free surrogate technique to identify cancer in its early stages.

Based on the testing and analysis of the different biological cell lines, the phase angles as functions of frequency that were plotted in FIG. 3 showed significant differences in frequencies. The impedance changes indicate that the cells have capacitive and resistive properties. In addition, these significant differences indicate that each cell line contains unique and relevant electrical information or bioelectric fingerprint. Statistical analysis using machine learning methods of the experimental data for the different cell line comparisons also revealed frequencies that contain the highest quality of information for cellular classification.

Embodiments of the disclosed microfluidic system improves bioelectric characterization of cells. Even though cell lines look very similar under a compound microscope, as well as the raw data plots, they clearly possess distinct bioelectrical signatures in several frequency ranges, which can be identified using various embodiments of the disclosed biodevice. Because the biodevice has a baseline impedance, fabrication parameters and techniques can affect how apparent cell differentiation in EIS outputs, and enhancing fabrication parameters and techniques can enhance data quality.

In various embodiments, thorough electrical recordings of other cell types can be obtained, such as viable blood cells, and different cancer cell lines. The potentiostat can be designed to allow real time monitoring of trapped cells to obtain better information of the data. In addition, different biodevice configurations can be implemented to measure the electrical impedance of target cells at a predetermined optimal frequency at real time with techniques such as flow cytometry. This device setup could include both configurations since a frequency sweep should be performed to find the optimal frequency to measure the impedance of single cells at a fixed frequency in real time. The device could also be modified by using different electrodes, such as Au/Cr electrodes, that can be deposited (using sputtering) as metal films on the side walls by a lift-off process through a negative photoresist. In example embodiments, this would enhance electric measurements and minimize background noise in EIS experimentation by positioning electrodes as close as possible to the target cells through this microfabrication technique. The classification model accuracies can be further optimized by fine tuning the RF parameters through parametrization as well as testing with considerable amounts of cell data, not used in the model generation.

C. Identification of Diseases in Liquid Biopsies (e.g. Lymphoma, Leukemia)

There is no single test that can be used to definitively diagnose cancer. Nevertheless, there are many lab tests or procedures that are used in a routine evaluation to detect the potential for cancer in a patient. A tissue biopsy is often the only way to know for sure that cancer is present in a patient. However, you need to know a priori that an anomalous tissue is present in the patient. In addition, tissue biopsies are often a highly invasive procedure. Liquid biopsies are test performed on bodily fluids, such as blood, to isolate cancer cells or look for small pieces of DNA/RNA released by tumor cells in the patient's body fluids. Liquid biopsies allow for multiple samples to be taken over time, can help detect cancer at an early stage, can provide info to help plan for personalized treatment and is minimally invasive.

Embodiments of the disclosed technology can be used in liquid biopsies. The technology can identify the type of cancer cell based on the electrical properties of individual cells through Electrochemical Impedance Spectroscopy (EIS) since the acquired data is of higher quality than the one generated by dielectrophoresis. This is so because the electrical data is acquired in several frequencies and can be used to detect subtle differences in the electrical properties of biological cells which are unique to each cell type based on experimental findings. When used together with a machine learning model, embodiments of the technology can classify cell types based on the raw electrical data acquired from the system. The technology can bring us a step closer to transform diagnosis and prognosis of cancer based on liquid biopsies.

D. Potency Assay for the Manufacturing of Cell-Based Therapies (e.g. CAR T Cells)

Cell based therapies involve using cells as a living drug. Cell-based therapies can rely on the immune system and can be referred to as immunotherapy. In immunotherapy, T-cells are genetically modified to augment their detection capabilities and attack anomalous or cancer cells in the body that would normally be hidden to the immune system. This genetic modification induced the expression of a Chimeric Antigen Receptor (CAR) on T-cells to be able to identify cancer cells that have developed the ability to pass as normal cells when interrogated by immune system cells. At present, the manufacturing process for immunotherapies is personalized, complicated, time consuming and very expensive. A goal of the scientific community is to develop a manufacturing process of CAR T-cells that would open this type of treatment to the masses. One of the issues of the CAR T-cell manufacturing process is that the medical community has not been able to agree on an assay that measures the efficacy or potency of the CAR T-cell treatment before it reaches the patient. Recent studies show that the potency of the CAR T-cell treatment is related to the CAR expression on T-cells (e.g., more CAR expression, more cytotoxic activity). Since the CAR expression changes the morphology of T-cells, CAR T-cells may be identifiable in a T-cell population based on EIS. Results show that T-cells and CAR T-cells have different bioelectric signatures and can thus be classified. Based on the sensitivity of the system, differences in the amount of CAR expression can be detected in CAR T-cells batches, which could be used as a potency assay as greater CAR expression often translates to improved cytotoxic activity and better treatment efficacy.

### E. Supplemental Information

### E1. Micro-Fab

Embodiments of the disclosed approach employ micro and nano fabrication methods to create new devices for the study and analysis of single cells on a chip. A microfluidic system can capture and electrically characterize individual cells. Various example devices allow for observation and understanding of the cell as a whole organism. In alternative embodiments, smaller dimensions, lower costs due to batch fabrication, incorporation of sensing, signal conditioning, superior functionality, and the ability to provide redundancy and enhanced reliability can be incorporated. Embodiments can thus provide a noninvasive, label free, low cost, and reliable system that can be an optimal option for early cancer detection.

### E2. EIS Technique

This technique is used mainly in the characterization and study of the electrophysiological properties of cells leading to differentiation between abnormal and normal cells. It is a very resourceful yet affordable way to characterize cells based on their electrical response to a determined frequency range.

### E3. Equivalent Circuit

These equivalent circuits can be expressed in individual component impedance and finally an addition of all of them. Electrodes, LSN, and PBS impedance are show as a follows:

$${Z}_{T o p} = {Z}_{B o t t o m} = {[ \frac{1}{{R}_{E L}} + {jwC}_{E L} ]}^{- 1} + {R}_{P B S} \qquad ( 3 )$$

$${Z}_{L S N} = {[ \frac{1}{{R}_{Pore}} + {jwC}_{LSN} ]}^{- 1} \qquad ( 4 )$$

$${Z}_{Si} = {[ \frac{1}{{R}_{P B S}} + {jwC}_{Si} ]}^{- 1} \qquad ( 5 )$$

$${Z}_{TotalwoC} = 2 \times {Z}_{T o p} + {Z}_{L S N} + {Z}_{Si} \qquad ( 6 )$$

where Z_{Top} and Z_{Bottom} are the impedances in the top and bottom chambers of the device, REL is the electrode resistor, CEL is the electrode capacitor, R_{PBS} is PBS resistance, Z_{LSN} is the LSN impedance, Z_{Si} is the silicon impedance, and Z_{Totalwoc} is the total impedance for the device without the captured cell. For the microfluidic system with a captured cell, the difference in the equations would be the cell impedance (Z_{Cell}), the LSN capacitor (C_{LSN}), and leak resistance (Rₗₑₐₖ), which appears due to the space between the micro-pore and the cell membrane. Equations 3 and 5 are similar to this equivalent circuit. The following mathematical equations were derived from the equivalent circuit for the captured cell to obtain the cell impedance:

$${Z}_{L - C} = {[ \frac{1}{{R}_{Leak}} + {jwC}_{LSN} + {Z}_{Cell} ]}^{- 1} \qquad ( 7 )$$

$${Z}_{TotalwC} = 2 \times {Z}_{T o p} + {Z}_{L - C} + {Z}_{Si} \qquad ( 8 )$$

To obtain closest cell impedance in this device, it was necessary to subtract the impedance without cell and the impedance with the trapped cell. The following mathematical equations were derived to obtain the cell impedance:

$${Z}_{Cell} = {Z}_{TotalwC} - {Z}_{TotalwoC} \qquad ( 9 )$$

$${Z}_{Cell} = ( 2 \times {Z}_{Top} + {Z}_{L - C} + {Z}_{Si} ) - ( 2 \times {Z}_{Top} + {Z}_{LSN} + {Z}_{Si} ) \qquad ( 10 )$$

$${Z}_{Cell} = ( {Z}_{L - C} ) - ( {Z}_{LSN} ) \qquad ( 11 )$$

$${Z}_{Cell - pore} = {[ \frac{1}{{R}_{Leak}} + {Z}_{Cell} ]}^{- 1} - {R}_{Pore} \qquad ( 12 )$$

Due to equivalent circuit similarities, a simple mathematical subtraction can be enough to obtain the impedance of the cell. Nevertheless, avoiding raw data alterations, a separate study was implemented to determine if there were variations on device runs that could affect cell impedance measurements. This could originate during the chip fabrication process, where some chips may have variations in low stress nitride (LSN) or silicon wafer thickness, altering chip impedance. Impedance variations were measured for eight different silicon chips at different voltages and days. The results were analyzed through principal components analysis (PCA) to determine whether the chip variations were relevant enough to alter cell impedance measurements. Thirty samples were prepared for each chip. The results of this experiment concluded that the handling and use of different silicon chips showed small variations that were not high enough to be seen in a PCA. In conclusion, performing the experiment on different days or using a different chip did not affect the observed cell impedance.

### E4. EIS Chip Variability

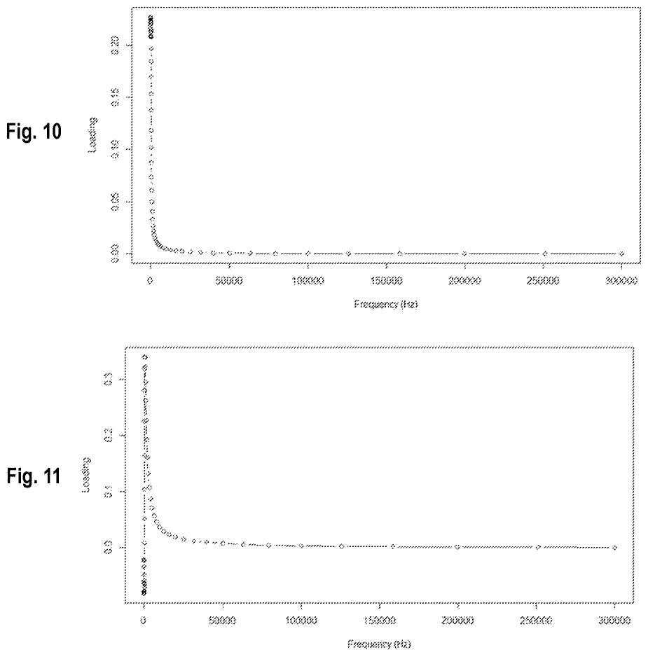

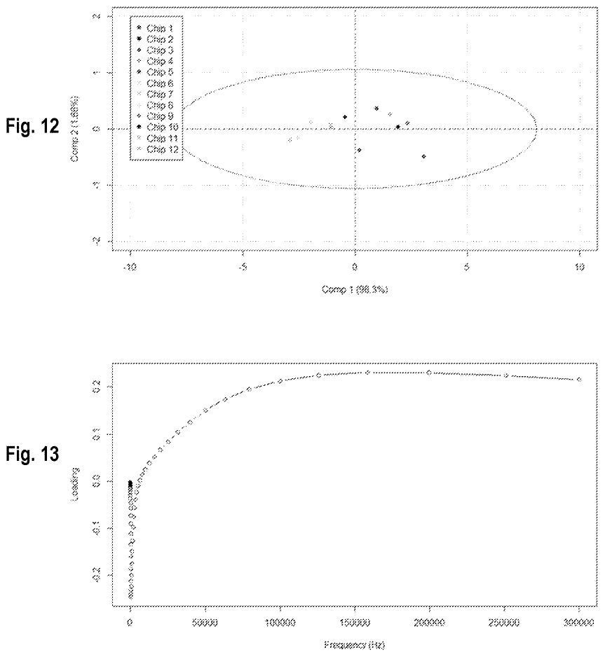

PCA was performed on the data obtained for the EIS experimentation on 12 individual chips to determine if different chips result in significant differences regarding the EIS data. The number of frequencies used in each EIS test was 56 and the total number samples for the 90 mV rms voltage was 1 per chip. The chips' impedance magnitude Loadings Line Plots for PC1 and PC2 were plotted to determine the influence of variation for each variable (frequencies). From FIGS. 10 and 11, no frequencies showed a variance correlation of 0.3 or higher for PC1, therefore no significant difference was obtained for the chips in the highest PC. PC2 showed frequencies with variance correlation higher than 0.3, thus the frequency range was reduced, and frequencies of interest were selected for further analysis and plotted as a Scores Plot, shown in FIG. 12. The data was scaled or normalized by removing the average and dividing the data by the standard deviation.

Deviation from theoretical data for the micro-pore resistance in the different chips could be attributed to imperfections in the micro-pore dimensions from the microfabrication process, which explains distancing in the data points for FIG. 12. In addition, an rms voltage was applied, which differs from theoretical measurements performed with DC voltage. Furthermore, the chips' phase angle Loadings Line Plots for PC1 and PC2 were also plotted to determine variations for each frequency. From FIGS. 13 and 14, no frequencies showed a variance correlation of 0.3 or higher for both PC1 and PC2, therefore no significant difference was obtained for the chips in the phase angle. It is important to note that phase angle shows highest differentiation patterns for the different biological cell lines, so the fact that there is no significant differentiation or variation between the different chips shows that the use of different chips does not affect the EIS data obtained.

### E5. Raw Data

Impedance magnitude and phase angle for each sample of HeLa, MCF12A (named as MCF), and MDA-MB-231 (named as MDA) cells lines (ATCC Laboratories) were exported as individual files using the EIS software of the GAMRY potentiostat (Gamry Instrument, Series G 300). Samples of the same cell line and EIS configuration were merged to generate an overall dataset per cell line and voltage. The code was made in MATLAB (MathWorks Inc.) and is found in https://github.com/MarcoBecerra15/EIS-Characterization-of-Cancer-Cells.-SI, the same link where all the codes to be mentioned can be found.

### E6. Outlier Screening

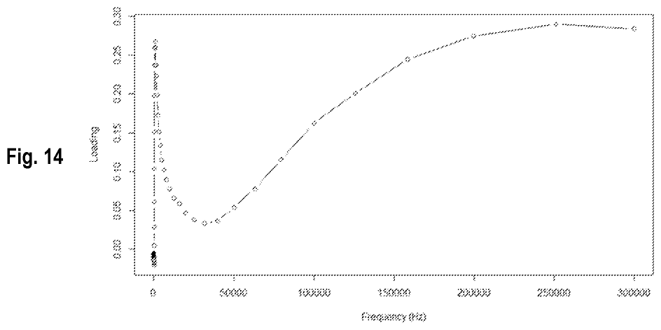

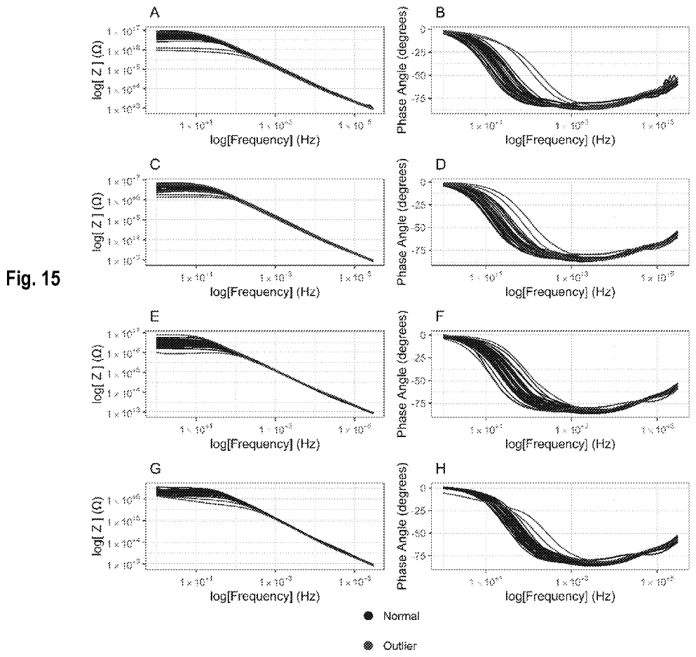

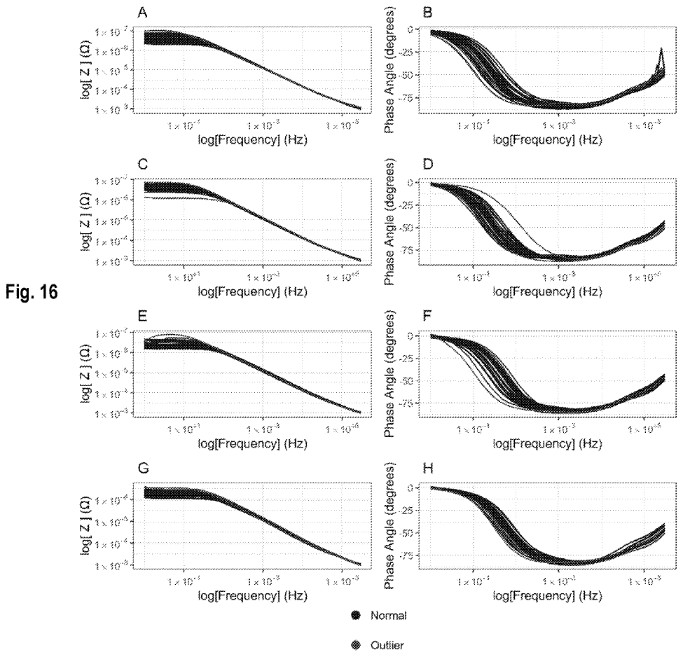

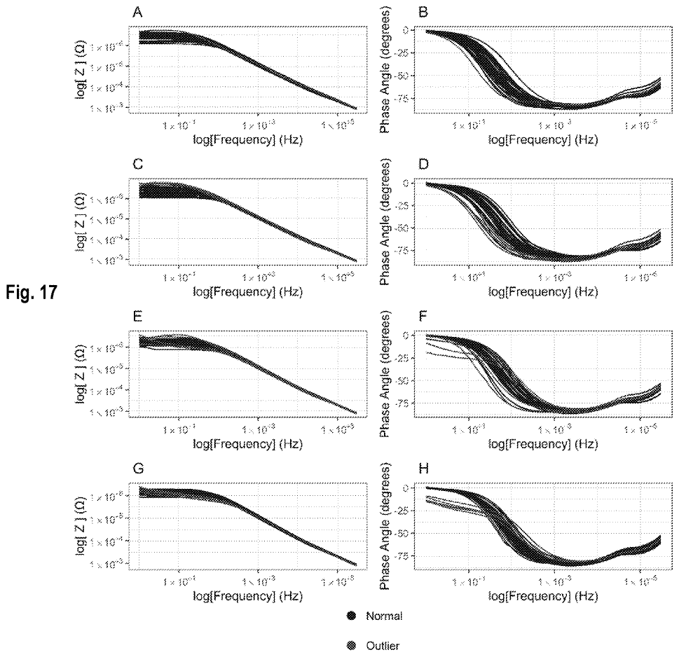

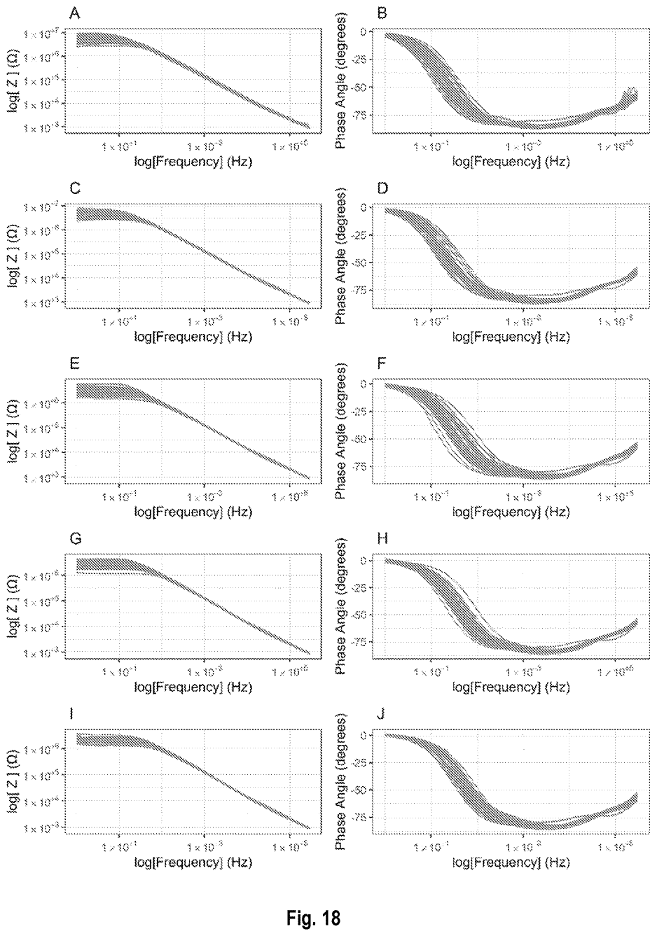

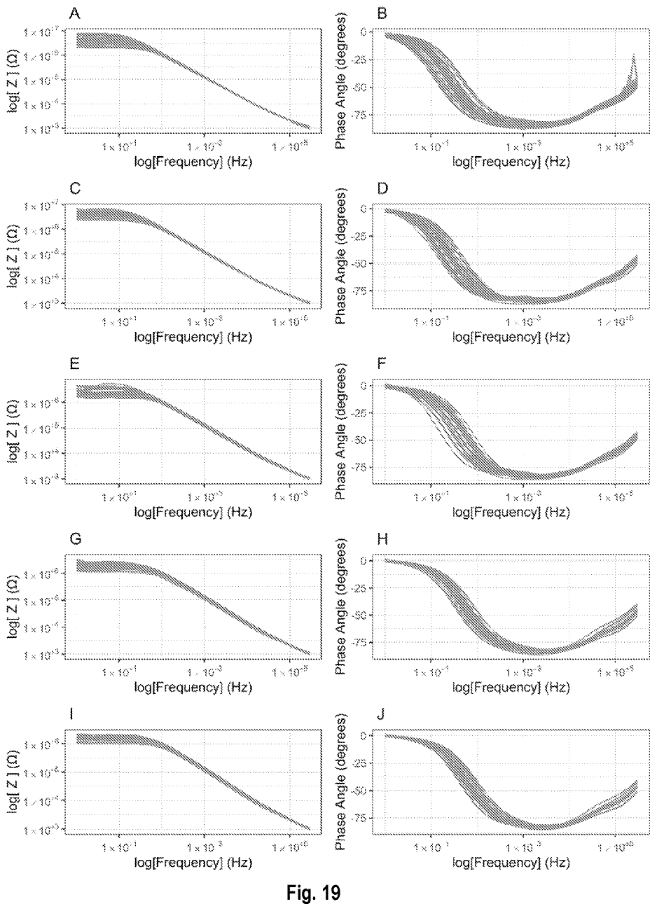

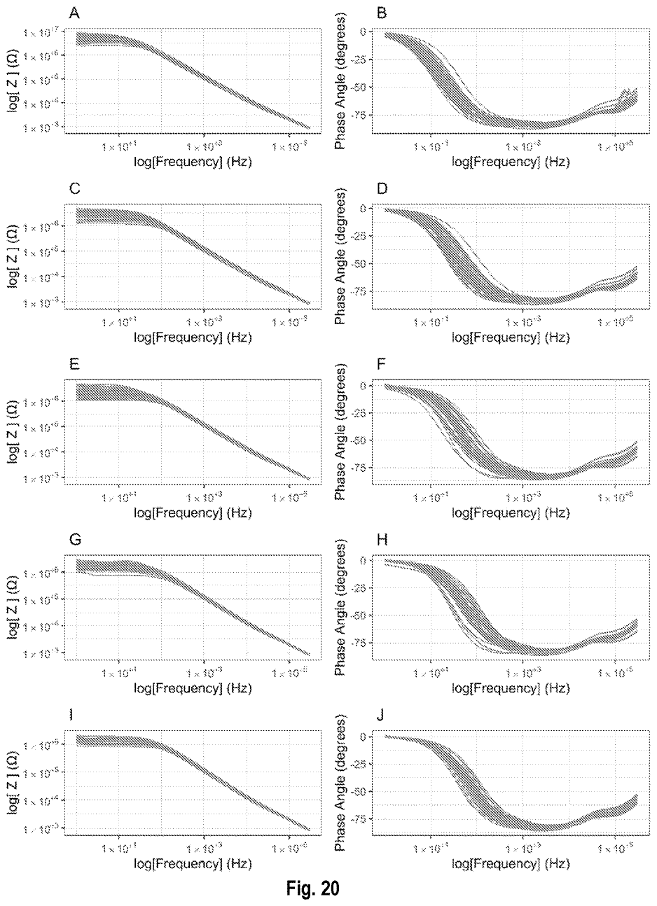

A filter for outliers was done by applying boxplot method frequency wise with all samples of the same cell line. However, samples were only discarded when they were deemed outliers in at least 75% of the tested frequencies. The code employed for this screening was also done in MATLAB. However, the plots that are presented in FIGS. 15-20 were made in R (R Core Team).

After this screening, the data sets were first merged to compare pairs of cell lines: MCF12A and MDA-MB-231, HeLa and MDA-MB-231, HeLa and MCF12A. Using the impedance phase angle and magnitude, labelled as Zphz and Zmod in the code, respectively, 6 data sets in total were obtained (Zmod HeLa, Zphz HeLa, Zmod MCF, Zphz MCF, Zmod MDA, and Zphz MDA). This merging was coded in MATLAB. Furthermore, separate datasets merging the 3 cell lines were made using R, and the same impedance components obtained from EIS.

### E7. Random Forests Models Generation

R Studio was used to generate the random forests models, applying the “randomForest” library. The code contains a line setting the random number seed at 42 precedes. This ensures only the desired parameters change in the next optimization steps, and not others like the samples that are selected during the bagging process. The error rate plot is then made and the total number of trees for the model is optimized. The number of variables considered at each node when building the decision trees is then optimized. A loop is run that iterates over this parameter, termed “p”, using the determined number of trees in the previous step. When the lowest error was obtained from different values of p, the highest was chosen arbitrarily. Then, a final call of the RF function was done, using both optimized parameters, in an effort of lower the out-of-bag error rate (OOBER) with respect to the initial call. The most important variables by randomization and multidimensional scaling (MDS) plot were then generated.

In R, the randomForests function was used. Two parameters were tuned to generate the model with the lowest possible Out of Bag Error Rate (OOBER): total number of trees in the model (ntree) and number of variables evaluated at each tree node (mtry). First, the ntree was tuned by selecting the value at which the OOBER achieves the lowest value possible. An initial value of 1000 is given in the first call, and the function records the OOBER at each ntree number until the specified amount. In cases where more than one ntree corresponds to the lowest OOBER, the highest is chosen. For example, if the OOBER stabilizes at 2% from ntree=700 onwards, 1000 is chosen. On the other hand, mtry was tuned by generating models with the identified optimal ntree from the previous step, but varying mtry. The one that yields the lowest OOBER is chosen. In case more than one value coincides, the highest is chosen. For example, having optimal ntree=500, models are made with mtry=1 onwards and ntree=500. If the lowest OOBER was 2%, but achieved at mtry =12, and 28, then 28 is chosen. The objective is to make the most accurate model possible with the EIS data, so the magnitude of these tuning parameters is not important. Models were made classifying between pairs of cell lines and between the 3 cell lines simultaneously. The model accuracy is deemed to be 100-OOBER.

Table 2 below shows the number of samples of each cell line the model incorrectly predicted at pairwise model generation. Table 3 shows the compilation of the confusion matrices for the 3-cell line models.

TABLE 2

| Impedance | Voltage | HeLa vs MCF | | HeLa vs MDA | | MCF vs MDA | |
|---|---|---|---|---|---|---|---|
| Component | (mV) | HeLa | MDA | MCF | MDA | HeLa | MCF |
| Phase | 30 | 0 | 0 | 0 | 0 | 0 | 1 |
| Angle | 60 | 0 | 0 | 2 | 2 | 0 | 1 |
| | 90 | 0 | 0 | 1 | 1 | 0 | 0 |
| | 120 | 0 | 0 | 1 | 2 | 0 | 0 |
| | 150 | 1 | 0 | 3 | 6 | 0 | 0 |
| Magnitude | 30 | 0 | 0 | 2 | 3 | 0 | 0 |
| | 60 | 0 | 0 | 3 | 1 | 0 | 0 |
| | 90 | 0 | 0 | 2 | 3 | 0 | 0 |
| | 120 | 0 | 0 | 0 | 2 | 0 | 0 |
| | 150 | 0 | 0 | 3 | 4 | 0 | 0 |

TABLE 3

| | Magnitude | | | | Phase Angle | | | |
|---|---|---|---|---|---|---|---|---|
| Voltage | Cell | | | | Cell | | | |
| (mV) | Lines | HeLa | MCF | MDA | Lines | HeLa | MCF | MDA |
| 30 | HeLa | 37 | 0 | 0 | HeLa | 36 | 0 | 1 |
| | MCF | 0 | 37 | 0 | MCF | 0 | 37 | 0 |
| | MDA | 0 | 1 | 36 | MDA | 1 | 0 | 36 |
| 60 | HeLa | 33 | 0 | 2 | HeLa | 33 | 0 | 2 |
| | MCF | 0 | 35 | 0 | MCF | 0 | 35 | 0 |
| | MDA | 2 | 1 | 32 | MDA | 1 | 0 | 34 |
| 90 | HcLa | 34 | 0 | 2 | HeLa | 36 | 0 | 0 |
| | MCF | 0 | 36 | 0 | MCF | 0 | 36 | 0 |
| | MDA | 1 | 0 | 35 | MDA | 2 | 0 | 34 |
| 120 | HeLa | 34 | 0 | 2 | HeLa | 35 | 0 | 1 |
| | MCF | 0 | 36 | 0 | MCF | 0 | 36 | 0 |
| | MDA | 1 | 0 | 35 | MDA | 0 | 0 | 36 |
| 150 | HeLa | 32 | 0 | 3 | HeLa | 33 | 0 | 2 |
| | MCF | 0 | 35 | 0 | MCF | 0 | 34 | 1 |
| | MDA | 6 | 0 | 29 | MDA | 3 | 0 | 32 |

### E8. Leave-One-Out Cross Validation (LOOCV)

The final models were then validated with Leave-One-Out Cross Validation (LOOCV), coded in R. As the method states, n-amount of RF models were generated, where each left out a different sample for that model to predict. The total number of samples in the comparison dataset is n. For example, the first model is made of all the samples except the 1st, which is in turn used to predict. The last model will not have sample n, but it will be used for prediction. The results of all the predictions are stored, and the total accuracy will be the number of correct predictions over the total number (n).

### E9. Principal Component Analysis (PCA)

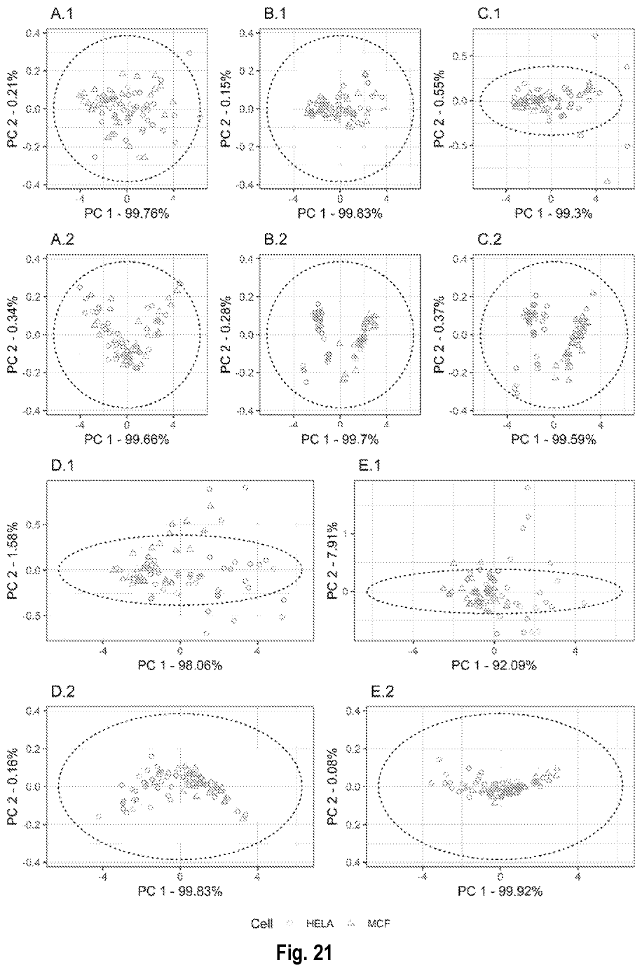

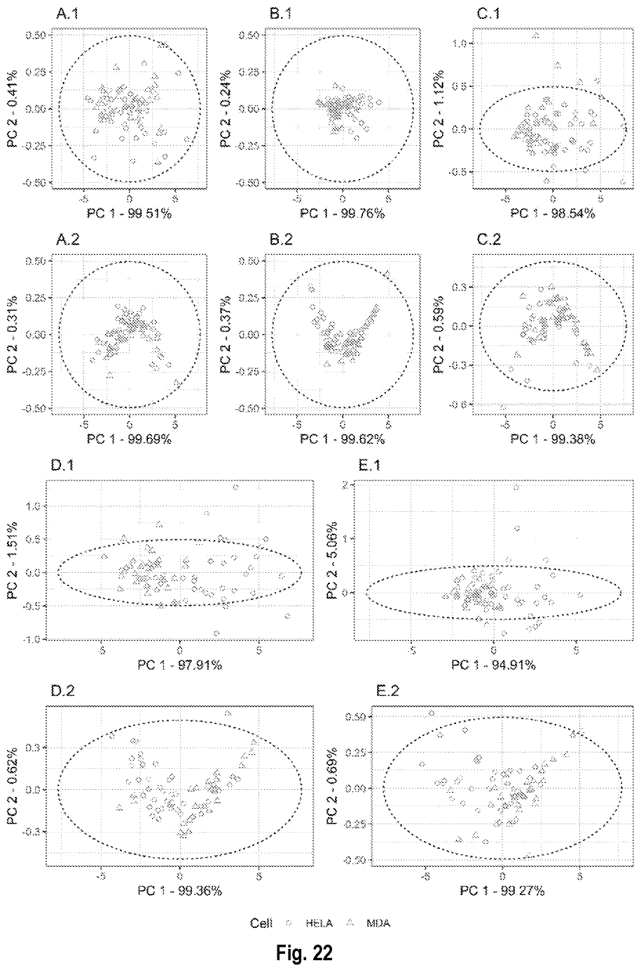

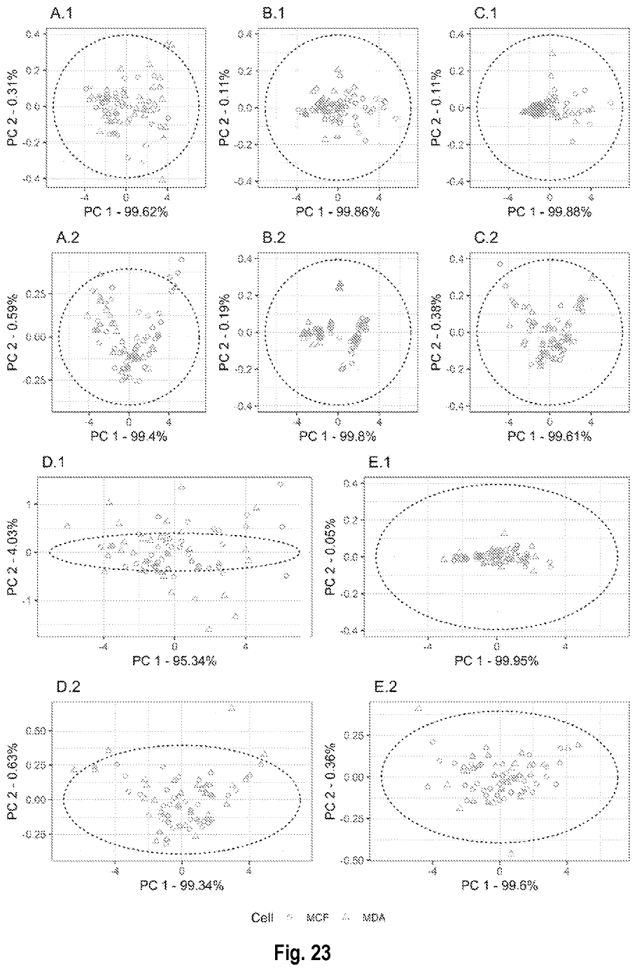

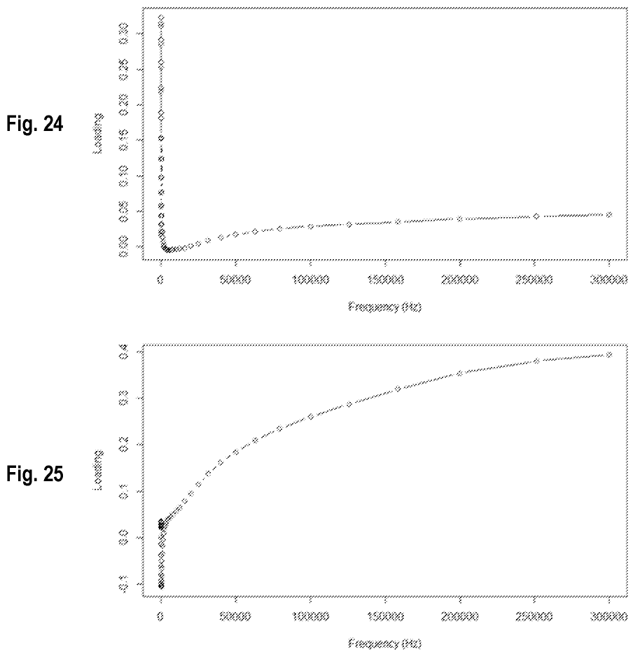

PCA was done in R using the pairwise data. A reduced dataset was made consisting of only the frequencies that scored the highest loadings in Principal Components 1 and 2, which amass the highest variation of the data. This selection was made using the Loadings Table the function generates. PCA was then run on this reduced set, which has the same samples as the original, but only the chosen frequencies. The Scores Plot for these datasets are presented. Principal Components 1 and 2 were plotted along with a 95% confidence ellipse. See FIGS. 21-23.

PCA was performed on the data obtained for the EIS experimentation on the HeLa, MDA-MB-231, and MCF12A cell lines. Prior to this study, confidence intervals were applied to the bioelectric spectra of the HeLa, MDA-MB-231, and MCF12A cell lines. The confidence intervals were made for each frequency where cell lines were evaluated, the number of frequencies used in each EIS test was 56 and the total number samples per voltage was 40. For the impedance magnitude and phase angle as a function of frequency, frequency ranges were selected for further analysis. These frequency ranges were selected based on the confidence intervals, making sure that frequencies where the confidence interval contained 0 was not selected, which means that it is not possible to differentiate one cell line from the other in any frequency. After selecting the appropriate frequency ranges, the data is processed, transformed, and presented in the PC plots using R. From the Loadings Line Plot, the influence of variation for each variable (frequencies) can be observed. From this plot, frequencies of interest were selected for further analysis, with a variance correlation of 0.3 or higher.

Reducing the frequency range, the selected frequencies were tested, and a Scores Plot was developed for the impedance magnitude and phase angle, respectively. The data was scaled or standardized by removing the baseline and dividing the data by the standard deviation. As previously mentioned, the Scores Plot describes the data structure in terms of sample patterns, showing correlation and variance between samples, as opposed to the Loading Line Plot, which showed variance between variables. In addition, a 90% confidence limit was used for the hoteling ellipses applied to the Scores Plots, which represents 90% of the explained variance in the data. The following plots are shown for 90 mVrms for all cell line comparisons.

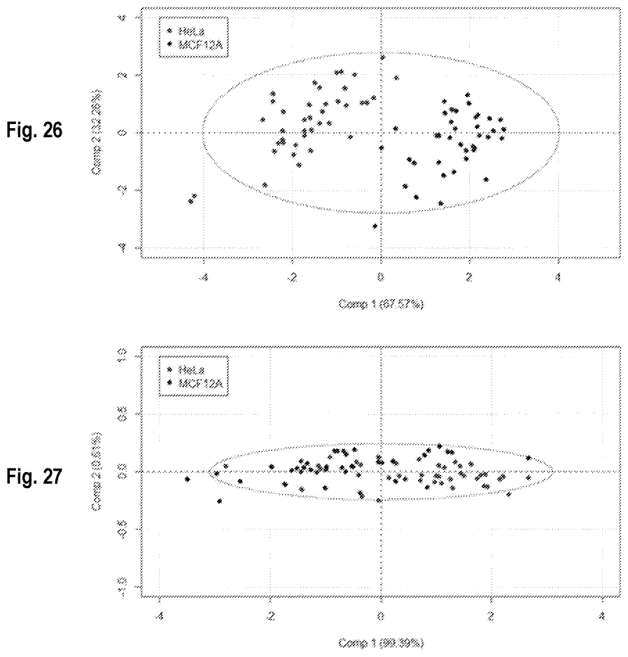

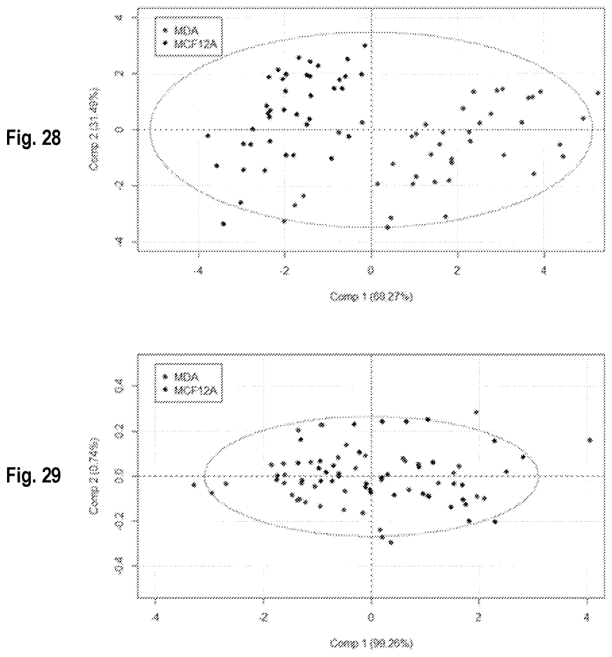

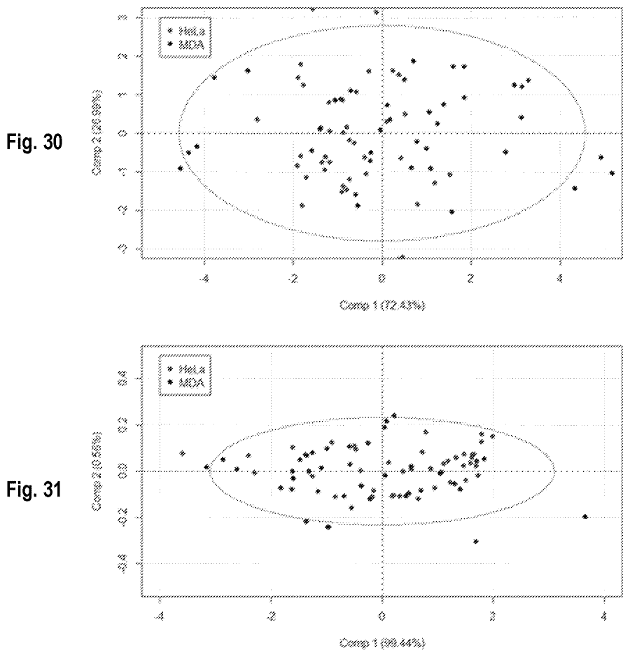

From FIGS. 26-31, a high differentiation can be observed for the selected frequencies in the phase angle for the HeLa/MCF12A and MDA-MB-231/MCF12A cell line comparisons. However, for the HeLa/MDA-MB-231 comparison, a low differentiation was observed. This can be due to the fact that in the frequencies selected for the analysis the cell lines do not express a sufficient differentiation or variance to be shown in the Scores Plot. Furthermore, the phase angle shows the highest level of differentiation for all cell lines, which expresses how much the imaginary component of the impedance is changing with respect to the real component.

While this specification contains many specific implementation details, these should not be construed as limitations on the scope of any implementations or of what may be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. Although the examples provided herein relate to controlling the display of content of information resources, the systems and methods described herein can include applied to other environments. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

Having described certain embodiments of methods and systems, it will now become apparent to one of skill in the art that other embodiments incorporating the concepts of the disclosure may be used. Therefore, the disclosure should not be limited to certain embodiments, but rather should be limited only by the spirit and scope of the following claims.

What is claimed is:

1. A method comprising:
receiving, by a computing device comprising one or more processors, bioelectrical data for a particle, the bioelectrical data comprising, or being based at least in part on, a plurality of impedance measurements;
determining, by the computing device, a property of the particle, wherein determining the property comprises inputting the bioelectrical data to a machine learning model; and
outputting, by the computing device, the property of the particle, wherein outputting the property comprises at least one of displaying the property on a display screen of the computing device, transmitting the property to another computing device via a network connection, or storing the property in a non-transitory computer-readable medium.

2. The method of claim 1, further taking measurements to obtain the bioelectrical data.

3. The method of claim 2, wherein taking measurements comprises applying an AC waveform to the particle.

4. The method of claim 2, wherein obtaining the bioelectrical data further comprises mechanically immobilizing the particle.

5. The method of claim 2, wherein the measurements are taken using one or more silver chloride (AgCl) electrodes.

6. The method of claim 1, wherein the plurality of impedance measurements comprises one or more phase angle measurements.

7. The method of claim 1, wherein the one or more impedance measurements comprises one or more impedance magnitude measurements.

8. The method of claim 1, wherein the plurality of impedance measurements comprises a plurality of phase angle measurements and a plurality of impedance magnitude measurements.

9. The method of claim 1, wherein the bioelectrical data is indicative of impedance of the particle at a plurality of discrete frequencies.

10. The method of claim 9, wherein each of the plurality of discrete frequencies is between 1 Hz and 1 MHz.

11. The method of claim 1, wherein the machine learning model is configured to receive, as inputs, impedance magnitude data and phase angle data, and provide, as an output, (i) the property or (ii) a classification indicative of the property.

12. The method of claim 1, wherein the machine learning model employs a random forest technique.

13. The method of claim 1, wherein the particle is a biological cell.

14. The method of claim 13, wherein the property corresponds to presence of a molecule in a membrane of the biological cell.

15. The method of claim 1, wherein the property is indicative of a presence of a tumor.

16. The method of claim 1, wherein the particle is a first particle of a first type, and wherein the property distinguishes the first particle from a second particle of a second type.

17. A computing device comprising one or more processors, the computing device being configured to:
receive bioelectrical data for a particle, the bioelectrical data comprising, or being based at least in part on, a plurality of impedance measurements;
determine a property of the particle, wherein determining the property comprises inputting the bioelectrical data to a machine learning model; and
output the property of the particle, wherein outputting the property comprises at least one of displaying the property on a display screen of the computing system, transmitting the property to another computing device via a network connection, or storing the property in a non-transitory computer-readable medium.

18. The computing device of claim 17, wherein the plurality of impedance measurements comprises impedance magnitude measurements and phase angle measurements.

19. The computing device of claim 17, wherein the bioelectrical data is indicative of impedance of the particle at a plurality of discrete frequencies.

20. The computing device of claim 17, wherein the machine learning model employs a random forest technique.
